(19) United States
(12) Patent Application Publication
(10) Pub. No.: US 2021/0106525 A1
(43) Pub. Date: Apr. 15, 2021

(54) FORMULATIONS FOR GASTROINTESTINAL DELIVERY OF OLIGONUCLEOTIDES

(71) Applicants: Massachusetts Institute of Technology, Cambridge, MA (US); The Brigham and Women's Hospital, Inc., Boston, MA (US)

(72) Inventors: Carlo Giovanni TRAVERSO, Newton, MA (US); Yunhua SHI, Belmont, MA (US); Thomas Christian VON ERLACH, Cambridge, MA (US); Robert S. LANGER, Newton, MA (US)

(21) Appl. No.: 17/066,534

(22) Filed: Oct. 9, 2020

Related U.S. Application Data
(60) Provisional application No. 62/914,048, filed on Oct. 11, 2019.

(51) Int. Cl. A61K 9/107; C12N 15/113; A61P 1/00

(52) U.S. Cl. CPC A61K 9/107; A61P 1/00; C12N 15/113

(57) ABSTRACT

Compositions and methods for effective delivery of oligonucleotide therapeutics, and in particular locked nucleic acid (AON)-containing gapmers, into the gastrointestinal (GI) tract are provided.

### CROSS-REFERENCE TO RELATED APPLICATIONS

This application claims the benefit of U.S. Provisional Patent Application Ser. No. 62/914,048, filed Oct. 11, 2019. The entire contents of which is incorporated herein by reference.

### REFERENCE TO SEQUENCE LISTING

The instant application contains a Sequence Listing which has been submitted electronically in ASCII format via EFS-Web and is hereby incorporated by reference in its entirety. Said ASCII copy, created Dec. 29, 2020, is named “MITN-047_Sequence-Listing.txt” and is 3610 bytes in size.

### BACKGROUND OF THE INVENTION

Therapeutic oligonucleotides have the theoretical capacity to regulate the expression of any gene and therefore could be applied for any drug target benefiting from modulation of expression. An antisense oligonucleotide (AON)-target interaction is based on the specific complementary targeting of a messenger RNA sequence of interest, which greatly increases the specificity and potency of oligonucleotide-based therapeutics as compared to small molecule drugs (Ming et al. (2011) Expert Opin. Drug. Deliv. 8:435-449; Vaishnaw et al. (2010) Silence 1:1-13). Therefore, orally-delivered oligonucleotides could have enormous therapeutic potential for a wide range of gastrointestinal related diseases. However, oligonucleotide-based therapeutics show low stability in the enzyme-rich GI tract, are unable to pass the mucus layer and show very poor GI absorption (Ensigna et al. (2012) Adv. Drug. Deliv. Rev. 64:557-570; Thomsen et al. (2014) Nanoscale 6:12547-12554).

Oligonucleotide-based therapeutics typically have been delivered intravenously, intraperitoneally or subcutaneously, and have been formulated in saline or buffered saline solutions, as well as being formulated into liposomes or nanoparticles (see e.g., Gao et al. (2009) Mol. Therap. 17:1225-1233 Seth et al. (2009) J. Med. Chem. 52:10-13; Obad et al. (2011) Nat. Genet. 43:371-378; Hildebrandt-Eriksen et al. (2012) Nucl. Acids Therap. 22:152-161; Thomas et al. (2012) RNA Biol. 9:1088-1098; Hagedorn et al. (2013) Nucl. Acid Therap. 23:302-310; Burdick et al. (2014) Nucl. Acids Res. 42:4882-4891; Kakiuchi-Kiyota et al. (2014) Toxicol. Sci. 138:234-248; Deng et al. (2015) Genet. Mol. Res. 14:10087-10095; Burel et al. (2016) Nucl. Acids Res. 44:2093-2109; Katsuya et al. (2016) Sci. Rep. 6:30377; Torres et al. (2016) BMC Cancer 16:822; Fernandez et al. (2018) Materials (Basel) 11: E122; Javanbakht et al. (2018) Mol. Ther. Nucl. Acids 11:441-454; US Patent Publication 20150299696; PCT Publication WO 2017/193087).

Compositions and methods for nonparental delivery of oligonucleotides, including buccal, sublingual, endoscopic, rectal, oral, vaginal, topical, pulmonary, or urethral delivery, also have been described (see e.g., US Patent Publication No. 20030040497; US Patent Publication No. 20040229831; US Patent Publication No. 20070249551; US Patent Publication No. 20130274309; and US Patent Publication No. 20160032289).

It has been reported that increased systemic bioavailability of orally administered AONs can be achieved using chemical enhancers that act as disruptors of the intestinal epithelial barrier such as sodium caprate (see e.g., Tillman et al. (2008) J. Pharm. Sci. 97:225-236; Aungst et al. (2012) AAPS J. 14:10-18; and US Patent Publication No. 20160032289). However, such an approach that results in disruption of the intestinal epithelium barrier is likely to have deleterious side effects.

Rationally designed nano- and micro formulations for local delivery of AONs to the GI tissue also have been reported (see e.g., Boirivant et al. (2006) Gastroenterology 131:1786-1798; Aouadi et al. (2009) Nature 458:1180-1184; Monteleone et al. (2015) N. Engl. J. Med. 372:1104-1113; Murakami et al. (2015) Sci. Rep. 5:1-13; Kang et al. (2017) ACA Nano 11:10417-10429; Ball et al. (2018) Sci. Rep. 8:1-12)

Additional formulations for oligonucleotide therapeutics that allow for effective delivery into the gastrointestinal tract are still needed.

### SUMMARY OF THE INVENTION

This disclosure provides formulations that allow for effective delivery of oligonucleotide therapeutics, including antisense oligonucleotides, such as locked nucleic acid-containing gapmers, into the gastrointestinal (GI) tract. Through systematic evaluation of a wide range of chemical compounds using an in vitro model system that replicates the complex cell architecture of the small intestine as well as the mucus layer, new GI mucosa uptake enhancers for use in oligonucleotide formulations have been identified that allow for efficacious delivery of oligonucleotides into the GI tract. These formulations can enhance gastrointestinal perfusion, gastrointestinal absorption or both gastrointestinal perfusion and absorption. In certain embodiments, the formulation comprises one or more compounds that enhance mucosal penetration, mucosal diffusion or both mucosal penetration and diffusion for local mucosal absorption and/or enhanced systemic bioavailability.

In one aspect, the disclosure pertains to compositions of an oligonucleotide and an oil formulated as an oil emulsion, wherein the oil emulsion enhance gastrointestinal delivery of the oligonucleotides. Accordingly, in one embodiment, the disclosure provides a composition for gastrointestinal delivery, the composition comprising: (i) at least one oligonucleotide; and (ii) at least one oil, formulated as an oil emulsion, wherein gastrointestinal delivery of the composition is greater than gastrointestinal delivery of the oligonucleotide alone.

In one embodiment, the oligonucleotide is an antisense oligonucleotide. In one embodiment, the antisense oligonucleotide is a locked nucleic acid (LNA) oligonucleotide. In one embodiment, the LNA oligonucleotide targets HIF-1 alpha. In one embodiment, the LNA oligonucleotide targets PTEN.

In one embodiment, the oil is selected from the group consisting of anise oil, cade oil, canola oil, Cassia oil, castor oil, celery oil, cinnamon oil, citronella oil, clove bud oil, coconut oil, corn oil, cottonseed oil, croton oil, cypress oil, Eucalyptus oil, fennel oil, flax seed oil, geranium oil, jojoba oil, lavender oil, lemon oil, mandarin oil, mineral oil, olive oil, peanut oil, rosemary oil, sandalwood oil, soya bean oil, thyme oil, tung oil, vegetable oil, wheatgerm oil and wintergreen oil. In one embodiment, the oil is corn oil, mineral oil or vegetable oil.

In one embodiment, the composition further comprises at least one emulsifier. In one embodiment, the emulsifier is selected from the group consisting of Soluplus®, Pluronic® F-127 and Tween® 20.

In one embodiment, gastrointestinal absorption of the composition is greater than gastrointestinal absorption of the oligonucleotide alone. In one embodiment, gastrointestinal perfusion of the composition is greater than gastrointestinal perfusion of the oligonucleotide alone. In one embodiment, both gastrointestinal absorption and perfusion of the composition is greater that that of the oligonucleotide alone.

In another aspect, the disclosure pertains to a composition of an oligonucleotide that comprises at least one gastrointestinal delivery enhancer (GDE), which can be a variety of different types of substances that enhance gastrointestinal delivery of oligonucleotides. Accordingly, in one embodiment, the disclosure provide a composition for gastrointestinal delivery, the composition comprising: (i) at least one oligonucleotide; and (ii) at least one gastrointestinal delivery enhancer (GDE) selected from the group consisting of calcium salts, potassium salts, sodium salts, ammonium salts, dicarboxylic acids, cholines, chlorides, amino sugars, fatty acids, parabens, buffering agents, clays and oils, wherein gastrointestinal delivery of the composition is greater than gastrointestinal delivery of the oligonucleotide alone.

In one embodiment, the GDE is a calcium salt. Non-limiting examples of calcium salts include calcium carbonate, calcium phosphate monobasic, calcium amorphous nanoparticles, calcium D-gluconate and alginic acid calcium.

In one embodiment, the GDE is a potassium salt. Non-limiting examples of potassium salts include potassium phosphate dibasic and potassium disulfide.

In one embodiment, the GDE is a sodium salt. Non-limiting examples of sodium salts include sodium metabisulfite, sodium azide, sodium perchlorate monohydrate and 3-(trimethylsilyl)-1-propanesulfonic acid sodium.

In one embodiment, the GDE is an ammonium salt. Non-limiting examples of ammonium salts include include ammonium iron citrate.

In one embodiment, the GDE is a dicarboxylic acid. Non-limiting examples of dicarboxylic acids include adipic acid.

In one embodiment, the GDE is a choline. Non-limiting examples of cholines include choline bitartrate.

In on embodiment, the GDE is a chloride. Non-limiting examples of chlorides include Tin (II) chloride.

In one embodiment, the GDE is an amino sugar. Non-limiting examples of amino sugars include meglumine.

In one embodiment, the GDE is a fatty acid. Non-limiting examples of fatty acids include octanoic acid and 4-ethyloctanoic acid.

In one embodiment, the GDE is a paraben. Non-limiting examples of paraben include methylparaben and ethyl paraben.

In one embodiment, the GDE is a buffering agent. Non-limiting examples of buffering agents include HEPES and Tris base.

In one embodiment, the GDE is a clay. Non-limiting examples of clays include kaolin.

In one embodiment, the GDE is an oil. Non-limiting examples of oils include corn oil or vegetable oil.

In one embodiment, the oligonucleotide is an antisense oligonucleotide. In one embodiment, the antisense oligonucleotide is a locked nucleic acid (LNA) oligonucleotide. In one embodiment, the LNA oligonucleotide targets HIF-1 alpha. In one embodiment, the LNA oligonucleotide targets PTEN.

In one embodiment, gastrointestinal absorption of the composition is greater than gastrointestinal absorption of the oligonucleotide alone. In one embodiment, gastrointestinal perfusion of the composition is greater than gastrointestinal perfusion of the oligonucleotide alone. In one embodiment, both gastrointestinal absorption and perfusion of the composition is greater that that of the oligonucleotide alone.

In another aspect, the disclosure pertains to methods of using the compositions of the disclosure. Accordingly, in one embodiment, the disclosure provides a method of enhancing delivery of an oligonucleotide to gastrointestinal tissue, the method comprising administering a composition of the disclosure to the gastrointestinal tissue.

In another aspect, the disclosure pertains to compositions for enhanced gastrointestinal delivery of specific locked nucleic acid (LNA)-containing gapmers. For example, in certain embodiments, the locked nucleic acid (LNA)-containing gapmer targets HIF-1 alpha (hypoxia-inducible factor-1 alpha). In certain embodiments, the locked nucleic acid (LNA)-containing gapmer targets PTEN (phosphatase and tensin homolog). In certain embodiments the HIF-1 alpha or PTEN LNA oligonucleotide is formulated with a compound that enhances gastrointestinal perfusion, gastrointestinal absorption or both gastrointestinal perfusion and absorption. In certain embodiments, the HIF-1 alpha or PTEN LNA oligonucleotide is formulated with a compound that enhances mucosal penetration, mucosal diffusion or both mucosal penetration and diffusion.

Accordingly, in one aspect, the disclosure pertains to a composition for gastrointestinal delivery, the composition comprising:
(a) a locked nucleic acid oligonucleotide that targets HIF-1 alpha; and
(b) a gastrointestinal perfusion or absorption enhancer selected from the group consisting of vegetable oil, 3-(Trimethylsilyl)-propanesulfonic acid sodium, 4-methyloctanoic acid; 8 arm PEG, advan hydrothane, alginic acid ammonium, alginic acid calcium, alginic acid potassium, benzophenone, beta-alanine, calcium D-gluconate, calcium phosphate amorphous nanopowder, calcium silicate, choline bitartarate, choline chloride, D(+) cellobiose, D(+) Trehalose dihydrate, ethylparaben, glycerin, glycerol phosphate calcium, hydroxyapatite, L-histidine, magnesium phosphate dibasic, methyl paraben, octanoic acid, paraffin wax, pentadecalactone, Pluronic® F-127, Poly(sodium) 4-styrene sulfonate, Poly(ethylene-co-glycidyl methacrylate), Poly(ethylene-co-vinyl-acetate), potassium disulfite, potassium gluconate, potassium phosphate dibasic, potassium pyrophosphate, potassium silicate, Sigma 7-9 (Tris base), silica gel, sodium dodecyl sulfate, sodium gluconate, sodium hyaluronate, Tin (II) chloride, xylitol, zinc acetate, 8 arm PEG, calcium D-gluconate, calcium phosphate monobasic, Koliphor® EL, paraffin wax, peanut oil, PEG 400 Da, potassium disulfite, sodium perchlorate monohydrate, sodium tartrate dibasic, sucrose octa-acetate, Tin (II) chloride and Tris (hydroxymethyl) aminomethane.

In certain embodiments of the HIF-1 alpha LNA composition, the gastrointestinal perfusion or absorption enhancer is selected from the group consisting of vegetable oil, calcium phosphate amorphous nanopowder, choline bitartarate, calcium phosphate monobasic, Tin (II) chloride, methylparaben, calcium D-gluconate, potassium disulfite, sodium perchlorate monohydrate, alginic acid calcium, Sigma 7-9 (Tris base), ethyl paraben, 3-(Trimethylsilyl)-1-propanesulfonic acid sodium and potassium phosphate dibasic.

In certain embodiments of the HIF-1 alpha LNA composition, the gastrointestinal perfusion or absorption enhancer is selected from the group consisting of calcium phosphate monobasic, Tin (II) chloride, methylparaben, calcium D-gluconate, potassium disulfite.

In certain embodiments of the HIF-1 alpha LNA composition, the gastrointestinal perfusion or absorption enhancer is selected from the group consisting of vegetable oil, calcium phosphate amorphous nanopowder and choline bitartarate.

In certain embodiments of the HIF-1 alpha LNA composition, the gastrointestinal perfusion enhancer is selected from the group consisting of 3-(Trimethylsilyl)-propanesulfonic acid sodium, 4-methyloctanoic acid; 8 arm PEG, advan hydrothane, alginic acid ammonium, alginic acid calcium, alginic acid potassium, benzophenone, beta-alanine, calcium D-gluconate, calcium phosphate amorphous nanopowder, calcium silicate, choline bitartarate, choline chloride, D(+) cellobiose, D(+) Trehalose dihydrate, ethylparaben, glycerin, glycerol phosphate calcium, hydroxyapatite, L-histidine, magnesium phosphate dibasic, methyl paraben, octanoic acid, paraffin wax, pentadecalactone, Pluronic® F-127, Poly(sodium) 4-styrene sulfonate, Poly(ethylene-co-glycidyl methacrylate), Poly(ethylene-co-vinyl-acetate), potassium disulfite, potassium gluconate, potassium phosphate dibasic, potassium pyrophosphate, potassium silicate, Sigma 7-9 (Tris base), silica gel, sodium dodecyl sulfate, sodium gluconate, sodium hyaluronate, Tin (II) chloride, xylitol and zinc acetate.

In certain embodiments of the HIF-1 alpha LNA composition, the gastrointestinal perfusion enhancer is selected from the group consisting of alginic acid calcium, Sigma 7-9 (Tris base), ethyl paraben, 3-(Trimethylsilyl)-1-propanesulfonic acid sodium and potassium phosphate dibasic.

In certain embodiments of the HIF-1 alpha LNA composition, the gastrointestinal absorption enhancer is selected from the group consisting of 8 arm PEG, calcium D-gluconate, calcium phosphate monobasic, Koliphor® EL, paraffin wax, peanut oil, PEG 400 Da, potassium disulfite, sodium perchlorate monohydrate, sodium tartrate dibasic, sucrose octa-acetate, Tin (II) chloride and Tris (hydroxymethyl) aminomethane. In certain embodiments, the gastrointestinal absorption enhancer is sodium perchlorate monohydrate.

In another aspect, the disclosure pertains to a composition for gastrointestinal delivery, the composition comprising:
(a) a locked nucleic acid oligonucleotide that targets HIF-1 alpha; and
(b) a gastrointestinal perfusion or absorption enhancer comprising an oil emulsion selected from the group consisting of:
   (i) Soluplus® emulsified with an oil selected from the group consisting of canola oil, Eucalyptus oil, castor oil, tung oil, mandarin oil, peanut oil, flax seed oil, Cassia oil, cade oil, citronella oil, coconut oil, thyme oil, lavender oil, cypress oil and clove bud oil; or
   (ii) Pluronic® F-127 emulsified with an oil selected from the group consisting of canola oil, olive oil, sandalwood oil, croton oil, mandarin oil and thyme oil; or
   (iii) Tween® 20 emulsified with an oil selected from the group consisting of sandalwood oil, canola oil, vegetable oil, thyme oil and lavender oil.

In another aspect, the disclosure pertains to a composition for gastrointestinal delivery, the composition comprising:
(a) a locked nucleic acid oligonucleotide that targets HIF-1 alpha; and
(b) a gastrointestinal mucus penetration or diffusion enhancer selected from the group consisting of sodium tartrate, calcium D-gluconate, zinc acetate, calcium phosphate amorphous nanopowder, calcium phosphate, caffeine, alpha cyclodextrin, potassium pyrophosphate and xylitol.

In certain embodiments of the HIF-1 alpha LNA composition, the gastrointestinal mucus penetration enhancer is selected from the group consisting of sodium tartrate, calcium D-gluconate, zinc acetate, calcium phosphate amorphous nanopowder and calcium phosphate.

In certain embodiments of the HIF-1 alpha LNA composition, the gastrointestinal mucus diffusion enhancer is selected from the group consisting of sodium tartrate, caffeine, alpha cyclodextrin, potassium pyrophosphate, xylitol, calcium D-gluconate, calcium phosphate amorphous nanopowder and calcium phosphate.

In yet another aspect, the disclosure pertains to a composition for gastrointestinal delivery, the composition comprising:
(a) a locked nucleic acid oligonucleotide that targets HIF-1 alpha; and
(b) a gastrointestinal perfusion or absorption enhancer selected from the group consisting of corn oil, vegetable oil, mineral oil, alpha cyclodextrin, potassium pyrophosphate, xylitol, calcium D-gluconate, calcium iodate, calcium phosphate, calcium citrate tetrahydrate, sodium glycholate, an oil emulsion comprising celery oil and Pluronic® F-127, D-mannitol, caffeine, choline chloride, potassium pyrophosphate, calcium phosphate dibasic, methyl paraben, an oil emulsion comprising clove bud oil and Soluplus®, and an oil emulsion comprising lemon oil and Tween® 20.

In certain embodiments of the HIF-1 alpha LNA compositions of the disclosure, the locked nucleic acid oligonucleotide that targets HIF-1 alpha comprises the nucleotide sequence shown in SEQ ID NO: 1.

In another aspect, the disclosure pertains to a composition for gastrointestinal delivery, the composition comprising:
(a) a locked nucleic acid oligonucleotide that targets PTEN; and
(b) a gastrointestinal perfusion or absorption enhancer selected from the group consisting of 2-butyloctanoic acid 4-methyl valeric acid, acetyl salicylic acid, adipic acid, alginic acid ammonium, alginic acid potassium, alpha D-glucose, aluminum hydroxide, aluminum oxide, ammonium aluminum sulfate dodecahydrate, ammonium carbonate, ammonium chloride, ammonium iron (III) citrate, beta-alanine, beta-cyclodextrin, calcium carbonate, calcium citrate, calcium fluoride, calcium iodate, calcium phosphate amorphous nanopowder, calcium phosphate dibasic, choline chloride, D(+) Trehalose dihydrate, corn oil, dodecanedoic acid, D-tryptophan, Dynasan® 118 microfine, edatate disodium, EDTA, ethyl formate, ethylparaben, EUDRAGIT® RL PO, glycerin, glycerol phosphate calcium, hydroxyapatite, hydroxymethyl polystyrene, Iron (III) chloride, Iron (III) oxide, Kaolin, Kollidon® 12PF, Kolliphor® EL, L-histidine, lithium hydroxide, magnesium carbonate, magnesium oxide, magnesium phosphate dibasic, magnesium sulfate, Meglumine, methyl paraben, PEG 400 Da, Pluronic® F-127, potassium bromide, potassium citrate tribasic, potassium disulfite, potassium gluconate, potassium nitrate, potassium phosphate (dibasic), potassium pyrophosphate, potassium silicate, pyridoxine, Sigma 7-9 (Tris base), sodium azide, sodium bicarbonate, sodium carbonate, sodium dodecyl sulfate, sodium fluoride, sodium gluconate, sodium hyaluronate, sodium hydroxide, sodium malonate, sodium metabisulfite, sodium perchlorate hydrate, sodium perchlorate monohydrate, sodium phosphate monobasic, sodium pyrophosphate, sodium sulfite, sodium tetraborate decahydrate, starch from corn, suberic acid, sucrose octa-acetate, Taurodeoxycholate, Tetrabutyl ammonium phosphate, Tris (hydroxymethyl) aminomethane, Trisodium citrate, turmeric, xylitol, 2-butyloctanoic acid, 2-hydroxy 2-methyl propiophenone, 3,4-dihydroxyl 1-phenyl alanine, 4-ethyloctanoic acid, 4-methylnonanoic acid, 4-methylvaleric acid, 8 arm PEG, alpha cyclodextrin, aluminum lactate, ammonium molybdate, calcium L-lactate hydrate, calcium phosphate monobasic, calcium silicate, D(+) cellobiose, EUDRAGIT® RS PO, gelatin from cold water fish skin, HEPES, Iron (II) D-gluconate, L-lysine, L-proline, manganese sulfate, mineral oil, octanoic acid, paraffin wax, peanut oil, PEG 20 kDa, PEG-block-PEG-block-PEG, pentadecalactone, Poly(ethylene glycol) diacrylate, Poly(sodium 4-styrene sulfonate), Poly(ethylene-co-glycidyl methacrylate), Poly(propyl glycol) diglycidyl ether, potassium carbonate, R(+)-Limonene, sodium salicylate, Terpin-4-ol and zinc carbonate.

In certain embodiments of the PTEN LNA composition, the gastrointestinal perfusion or absorption enhancer is selected from the group consisting of calcium carbonate, adipic acid, Kaolin, ammonium iron citrate, sodium metabisulfite, HEPES, corn oil, 4-ethyloctanoic acid, calcium phosphate monobasic, octanoic acid, sodium azide, sodium perchlorate monohydrate, potassium phosphate (dibasic), Sigma 7-9 (Tris base) and Meglumine.

In certain embodiments of the PTEN LNA composition, the gastrointestinal perfusion or absorption enhancer is selected from the group consisting of calcium carbonate, adipic acid, Kaolin, ammonium iron citrate and sodium metabisulfite.

In certain embodiments of the PTEN LNA composition, the gastrointestinal perfusion enhancer is selected from the group consisting of 2-butyloctanoic acid 4-methyl valeric acid, acetyl salicylic acid, adipic acid, alginic acid ammonium, alginic acid potassium, alpha D-glucose, aluminum hydroxide, aluminum oxide, ammonium aluminum sulfate dodecahydrate, ammonium carbonate, ammonium chloride, ammonium iron (III) citrate, beta-alanine, beta-cyclodextrin, calcium carbonate, calcium citrate, calcium fluoride, calcium iodate, calcium phosphate amorphous nanopowder, calcium phosphate dibasic, choline chloride, D(+) Trehalose dihydrate, dodecanedoic acid, D-tryptophan, Dynasan® 118 microfine, edatate disodium, EDTA, ethyl formate, ethylparaben, EUDRAGIT® RL PO, glycerin, glycerol phosphate calcium, hydroxyapatite, hydroxymethyl polystyrene, Iron (III) chloride, Iron (III) oxide, Kaolin, Kollidon® 12PF, Kolliphor® EL, L-histidine, lithium hydroxide, magnesium carbonate, magnesium oxide, magnesium phosphate dibasic, magnesium sulfate, Meglumine, methyl paraben, PEG 400 Da, Pluronic® F-127, potassium bromide, potassium citrate tribasic, potassium disulfite, potassium gluconate, potassium nitrate, potassium phosphate (dibasic), potassium pyrophosphate, potassium silicate, pyridoxine, Sigma 7-9 (Tris base), sodium azide, sodium bicarbonate, sodium carbonate, sodium dodecyl sulfate, sodium fluoride, sodium gluconate, sodium hyaluronate, sodium hydroxide, sodium malonate, sodium metabisulfite, sodium perchlorate hydrate, sodium perchlorate monohydrate, sodium phosphate monobasic, sodium pyrophosphate, sodium sulfite, sodium tetraborate decahydrate, starch from corn, suberic acid, sucrose octa-acetate, Taurodeoxycholate, Tetrabutyl ammonium phosphate, Tris (hydroxymethyl) aminomethane, Trisodium citrate, turmeric and xylitol.

In certain embodiments of the PTEN LNA composition, the gastrointestinal perfusion enhancer is selected from the group consisting of sodium azide, sodium perchlorate monohydrate, potassium phosphate (dibasic), Sigma 7-9 (Tris base) and Meglumine.

In certain embodiments of the PTEN LNA composition, the gastrointestinal absorption enhancer is selected from the group consisting of 2-butyloctanoic acid, 2-hydroxy 2-methyl propiophenone, 3,4-dihydroxyl 1-phenyl alanine, 4-ethyloctanoic acid, 4-methylnonanoic acid, 4-methylvaleric acid, 8 arm PEG, acetyl salicylic acid, adipic acid, alginic acid ammonium, alginic acid potassium, alpha cyclodextrin, alpha D-glucose, aluminum hydroxide, aluminum lactate, aluminum oxide, ammonium aluminum sulfate dodecahydrate, ammonium carbonate, ammonium chloride, ammonium iron (III) citrate, ammonium molybdate, beta-cyclodextrin, calcium carbonate, calcium citrate, calcium fluoride, calcium iodate, calcium L-lactate hydrate, calcium phosphate amorphous nanopowder, calcium phosphate dibasic, calcium phosphate monobasic, calcium silicate, choline chloride, D(+) cellobiose, corn oil, dodecanedoic acid, D-tryptophan, Dynasan® 118 microfine, edatate disodium, EDTA, ethyl formate, EUDRAGIT® RL PO, EUDRAGIT® RS PO, gelatin from cold water fish skin, glycerol phosphate calcium, HEPES, hydroxyapatite, Iron (III) chloride, Iron (II) D-gluconate, Kaolin, Kolliphor® EL, L-histidine, lithium hydroxide, L-lysine, L-proline, magnesium carbonate, magnesium oxide, magnesium phosphate dibasic, magnesium sulfate, manganese sulfate, methyl paraben, mineral oil, octanoic acid, paraffin wax, peanut oil, PEG 20 kDa, PEG 400 Da, PEG-block-PEG-block-PEG, pentadecalactone, Pluronic® F-127, Poly(ethylene glycol) diacrylate, Poly(sodium 4-styrene sulfonate), Poly(ethylene-co-glycidyl methacrylate), Poly(propyl glycol) diglycidyl ether, potassium bromide, potassium carbonate, potassium citrate tribasic, potassium disulfite, potassium gluconate, potassium nitrate, potassium pyrophosphate, potassium silicate, pyridoxine, R(+)-Limonene, sodium bicarbonate, sodium carbonate, sodium fluoride, sodium gluconate, sodium hyaluronate, sodium hydroxide, sodium malonate, sodium metabisulfite, sodium perchlorate hydrate, sodium pyrophosphate, sodium salicylate, sodium sulfite, sodium tetraborate decahydrate, starch from corn, suberic acid, sucrose octa-acetate, Terpin-4-ol, Tetrabutyl ammonium phosphate, Tris (hydroxymethyl) aminomethane, turmeric, xylitol and zinc carbonate.

In certain embodiments of the PTEN LNA composition, the gastrointestinal absorption enhancer is selected from the group consisting of HEPES, corn oil, 4-ethyloctanoic acid, calcium phosphate monobasic and octanoic acid.

In another aspect, the disclosure pertains to a composition for gastrointestinal delivery, the composition comprising:
(a) a locked nucleic acid oligonucleotide that targets PTEN; and
(b) a gastrointestinal perfusion or absorption enhancer comprising an oil emulsion selected from the group consisting of:
   (i) Soluplus® emulsified with an oil selected from the group consisting of canola oil, jojoba oil, cinnamon oil, Eucalyptus oil, tung oil, fennel oil, peanut oil, Cassia oil, cade oil, thyme oil, lavender oil, mineral oil, mandarin oil, wintergreen oil, cypress oil, clove bud oil and cottonseed oil; or
   (ii) Pluronic® F-127 emulsified with an oil selected from the group consisting of celery seed oil, tung oil, citronella oil and cade oil; or
   (iii) Tween® 20 emulsified with an oil selected from the group consisting of Eucalyptus oil, geranium oil, epoxidized soya bean oil, olive oil, croton oil, anise oil, lemon oil, flax seed oil, wheat germ oil and rosemary oil.
In yet another aspect, the disclosure pertains to a composition for gastrointestinal delivery, the composition comprising:
(a) a locked nucleic acid oligonucleotide that targets PTEN; and
(b) a gastrointestinal mucus penetration or diffusion enhancer selected from the group consisting of sodium tartrate, D-mannitol, caffeine, alpha cyclodextrin, choline bitartarate, choline chloride, alginic acids, calcium citrate, calcium phosphate, potassium pyrophosphate and calcium D-gluconate.

In certain embodiments of the PTEN LNA composition, the gastrointestinal mucus penetration enhancer is selected from the group consisting of sodium tartrate, D-mannitol, caffeine, alpha cyclodextrin, choline bitartarate, choline chloride, alginic acids, calcium citrate and calcium phosphate.

In certain embodiments of the PTEN LNA composition, the gastrointestinal mucus diffusion enhancer is selected from the group consisting of sodium tartrate, potassium pyrophosphate, calcium D-gluconate and calcium phosphate.

In yet another aspect, the disclosure pertains to a composition for gastrointestinal delivery, the composition comprising:
(a) a locked nucleic acid oligonucleotide that targets PTEN; and
(b) a gastrointestinal perfusion or absorption enhancer selected from the group consisting of corn oil, vegetable oil, mineral oil, alpha cyclodextrin, potassium pyrophosphate, calcium iodate, calcium phosphate, sodium tartrate, xylitol, calcium D-gluconate, D-mannitol, sodium glycholate and an oil emulsion comprising celery oil and Pluronic® F-127.

In certain embodiments of the PTEN LNA compositions of the disclosure, the locked nucleic acid oligonucleotide that targets PTEN comprises the nucleotide sequence shown in SEQ ID NO: 3 or 4.

Methods of enhancing delivery of locked nucleic acid oligonucleotides to gastrointestinal tissue are also provided. For example, in one embodiment, the disclosure pertains to a method of enhancing delivery of a locked nucleic acid oligonucleotide that targets HIF-1 alpha to gastrointestinal tissue, the method comprising administering any of the HIF-1 alpha LNA-containing compositions of the disclosure to the gastrointestinal tissue. In another embodiment, the disclosure pertains to a method of enhancing delivery of a locked nucleic acid oligonucleotide that targets PTEN to gastrointestinal tissue, the method comprising administering any one the PTEN LNA-containing compositions of the disclosure to the gastrointestinal tissue. The methods of the disclosure for enhancing delivery of an LNA to gastrointestinal tissue can be used in a wide variety of clinical conditions relating to the gastrointestinal tract, as described herein.

These and other aspects and embodiments will be described in greater detail herein.

Each of the limitations of the invention can encompass various embodiments of the invention. It is therefore anticipated that each of the limitations of the invention involving any one element or combinations of elements can be included in each aspect of the invention. This invention is not limited in its application to the details of construction and/or the arrangement of components set forth in the following description or illustrated in the drawings. The invention is capable of other embodiments and of being practiced or of being carried out in various ways.

### BRIEF DESCRIPTION OF THE FIGURES

FIGS. 1A-1B are graphs showing results from a kinetic perfusion analysis of FAM-labelled locked nucleic acid (AON)-containing gapmers against either HIF-1 alpha (FIG. 1A) or PTEN (FIG. 1B).

FIGS. 2A-2B are graphs showing the linear correlation of intestinal tissue accumulation of locked nucleic acids (AON)-containing gapmers against either HIF-1 alpha (FIG. 2A) or PTEN (FIG. 2B) as measured by confocal microscopy-based detection versus spectrophotometric detection.

FIG. 3 is a graph showing the results of a variability analysis of FAM fluorescence signal of basal and apical small intestinal tissue incubated with locked nucleic acids (AON)-containing gapmers against either HIF-1 alpha or PTEN, as well as FAM only as a control, in various concentrations (n=192-288).

FIG. 4 shows a heatmap summary of the results of screening a panel of AON formulations for intestinal perfusion, apical absorption and basal absorption for locked nucleic acids (AON)-containing gapmers against either HIF-1 alpha or PTEN. Results are summarized as fold changes compared to the non-formulated control in a color-coded heatmap that shows permeability as well as absorption for the two AONs tested side-by-side. Results are shown for single excipient solution formulations screened using a custom designed library of 285 compounds from diverse chemical properties.

FIG. 5 shows a heatmap summary of the results of screening a panel of AON formulations for intestinal perfusion, apical absorption and basal absorption for locked nucleic acids (AON)-containing gapmers against either HIF-1 alpha or PTEN. Results are summarized as fold changes compared to the non-formulated control in a color-coded heatmap that shows permeability as well as absorption for the two AONs tested side-by-side. Results are shown for 213 oil-emulsion formulations for the two AONs tested (71 different organic oils were combined with 3 different emulsifiers: Soluplus®, Pluronic F127 and Tween 20).

FIG. 6 shows representative images of FAM fluorescence intensity of FAM-LAN (HIF-1 alpha) and FAM-LAN (PTEN) formulations placed on top of mucus layer and incubated for 75 minutes. Fluorescence signal displacement was used to assess diffusion of FAM-AON into the mucus layer.

FIG. 7 shows a heatmap summary of the results of screening a subpanel of formulations with FAM-LAN (HIF-1 alpha) or FAM-LAN (PTEN) for mucus diffusion as analyzed by 4D imaging. The results were compared to the change in intestinal permeability and absorption using the GIT-ORIS system with intestinal mucus layer intact versus washed away. The results are summarized as fold changes compared to the non-formulated control in a color-coded heatmap.

FIG. 8 shows a heatmap summary of the results of a panel of AON formulations for intestinal perfusion, apical absorption and basal absorption for locked nucleic acids (AON)-containing gapmers against either HIF-1 alpha or PTEN labeled with Alexa647. Results are averaged from 3 independent experiments, n=3.

FIG. 9 shows the expression analysis of the target genes PTEN and HIF-1 alpha in various porcine derived gastrointestinal segments.

FIG. 10 shows a heatmap summary of the knock-down efficiency of various formulations with the locked nucleic acids (AON)-containing gapmers against the target PTEN and HIF-1 alpha. Results are shown as a percentage of expression level of the target gene in the non-treated condition (n=4).

FIG. 11 shows photographs of in situ hybridization analysis of biopsy samples obtained from pig small intestine tissue exposed to different HIF-1 alpha targeting locked nucleic acids (AON)-containing gapmers formulations over a period of 1 hours using in vivo pig system described herein. Blue=DAPI, Green=AON signal. Scale bar=500 μm.

FIG. 12 is a graph showing the in vivo knock-down efficiency of various formulations with the locked nucleic acids (AON)-containing gapmers against HIF-1 alpha using the in vivo pig system described herein. Results are shown as a percentage of expression level of the target gene in the non-treated condition. Results show average of 3 independent experiments. Error bars show standard deviation. ** p<0.01, *** p<0.001.

### DETAILED DESCRIPTION OF THE INVENTION

Antisense oligonucleotides (AONs) have the potential to transform the ability to modulate gene expression for effective disease management. Oral AON delivery has the advantage of ease of administration as well as direct access to the gastrointestinal (GI) tract for topical treatment of a wide range of GI related diseases (see e.g., Dabaja et al. (2004) Cancer 101:518-526; Akhtar et al. (2009) J. Drug Target. 17:491-495; Baumgart et al. (2012) Lancet 380:1590-1605; Brenner et al. (2014) Lancet 383:1490-1502; Monteleone et al. (2015) N. Engl. J. Med. 372:1104-1113; Mojibian et al. (2016) J. Diabetes Investig. 7:87-93). However, low intestinal absorption has limited their administration to parenteral routes (see e.g., Goldberg et al. (2003) Nat. Rev. Drug Discov. 2:289-295; Ensigna et al. (2012) Adv. Drug Deliv. Rev. 64:557-570).

The present disclosure describes the development of an automated high throughput system that enables simultaneous modeling of permeability and tissue accumulation in porcine derived GI tract explants. Systematic screening of locked nucleic acids (AON)-containing gapmer formulation libraries on this system, revealed a wide range of novel formulations for potential topical or systemic oral delivery of AONs. Based on these results, AON nanoparticles and nanoaggregates have been identified that enable significant efficacy in vivo in pigs after just one hour of exposure in the GI tract without disruption of the epithelium. Accordingly, the compositions and methods of the disclosure can be used to significantly improve oral delivery of AONs and other oligonucleotides, including those comprising naturally-occurring nucleotides and those comprising non-naturally occurring nucleotides (e.g., nucleotide analogues), or a combination of both.

I. Oil Emulsion Formulations

As described in the Examples, oligonucleotide formulations comprising oil emulsions have been found to exhibit enhanced gastrointestinal delivery of the oligonucleotide (e.g., LNA-containing gapmer), as compared to delivery of the oligonucleotide alone (i.e., in the absence of the oil emulsion). Accordingly, in one aspect, the disclosure pertains to a composition for gastrointestinal delivery, the composition comprising: (i) an oligonucleotide; (ii) an oil formulated as an emulsion, wherein gastrointestinal delivery of the composition is greater than gastrointestinal delivery of the oligonucleotide alone.

In some embodiments, the oil emulsion is 70-85% oil and 15-30% aqueous buffer. In some embodiments, the oil emulsion is 80-85% oil and 15-20% aqueous buffer.

Non-limiting examples of oils that can be used in the composition include anise oil, cade oil, canola oil, Cassia oil, castor oil, celery oil, cinnamon oil, citronella oil, clove bud oil, coconut oil, corn oil, cottonseed oil, croton oil, cypress oil, Eucalyptus oil, fennel oil, flax seed oil, geranium oil, jojoba oil, lavender oil, lemon oil, mandarin oil, mineral oil, olive oil, peanut oil, rosemary oil, sandalwood oil, soya bean oil, thyme oil, tung oil, vegetable oil, wheatgerm oil and wintergreen oil. In certain embodiments, the oil is selected from the group consisting of corn oil, mineral oil or vegetable oil. In one embodiment, the oil is corn oil. In one embodiment, the oil is mineral oil. In one embodiment, the oil is vegetable oil.

Other no-limiting examples of oils include bay oil, canola oil, soybean oil, lovage oil, dillweed oil, cardamom oil, lemongrass oil, tea tree oil, jojoba oil from Simmondsia chinensis, cinnamon oil (ceylon type, nature identical), Eucalyptus oil, garlic oil (chinese), coriander oil, cognac oil, celery seed oil, corn oil, cedar oil, lard oil, bergamot oil, palm oil, castor oil, guaiac wood oil, ginger oil, geranium oil (chinese), nutmeg oil, peppermint oil, epoxidized soya bean oil, wheat germ oil, palm fruit oil, jojoba oil, tung oil, sandalwood oil, fennel oil, olive oil, linseed oil, menhaden fish oil, croton oil, peanut oil, anise oil, coffee oil, fusel oil, patchouli oil, lemon oil, spearmint oil, vegetable oil, sesame oil, flax seed oil, rosemary oil, mandarin oil, Cassia oil, cade oil, citronella oil (java), coconut oil, safflower oil, sunflower seed oil, clove oil, rapeseed oil from Brassica rapa, cedar leaf oil, avocado oil, thyme oil, lavender oil, orange oil, mineral oil, sunflower oil, wintergreen oil, lime oil, pine needle oil, birch oil, cypress oil, clove bud oil and cottonseed oil.

In one embodiment, the composition further comprises an emulsifier, also referred to as an emulsifying agent. The emulsifier aids in stabilizing the mixture of the oligonucleotide and the oil. Emulsifiers typically have a polar or hydrophilic (i.e., water soluble) part and a non-polar (i.e., hydrophobic or lipophilic) part. In one embodiment, the emulsifier is a surfactant. In one embodiment, the emulsifier is a detergent. In one embodiment, the emulsifier is selected from the group consisting of Soluplus®, Pluronic® F-127 and Tween® 20, each of which is commercially available. Other non-limiting examples of emulsifiers include lecithin, TritonX100, Tween® 80, Tween® 28, and Span® 80.

In certain embodiments, the composition can comprise any of the following combinations of emulsifiers and oils:
(i) Soluplus® emulsified with an oil selected from the group consisting of cade oil, Cassia oil, canola oil, castor oil, cinnamon oil, citronella oil, clove bud oil, coconut oil, cottonseed oil, cypress oil, Eucalyptus oil, flax seed oil, fennel oil, jojoba oil, lavender oil, mandarin oil, mineral oil, peanut oil, thyme oil, tung oil and wintergreen oil; or
(ii) Pluronic® F-127 emulsified with an oil selected from the group consisting of cade oil, canola oil, celery oil, citronella oil, croton oil, mandarin oil, olive oil, sandalwood oil, thyme oil and tung oil; or
(iii) Tween® 20 emulsified with an oil selected from the group consisting of anise oil, canola oil, croton oil, Eucalyptus oil, flax seed oil, geranium oil, lavender oil, lemon oil, olive oil, rosemary oil, sandalwood oil, soya bean oil (e.g., epoxidized soya bean oil), thyme oil, vegetable oil and wheat germ oil.

The oligonucleotide compositions comprising an oil emulsion can be prepared by standard methods known in the art, such as described in the Examples.

In one embodiment, the oligonucleotide is an antisense oligonucleotide (e.g., antisense RNA). In one embodiment, the antisense oligonucleotide comprises at least one locked nucleic acid (LNA), referred to herein as an LNA oligonucleotide. In one embodiment, the LNA oligonucleotide targets HIF-1 alpha. In one embodiment, the LNA oligonucleotide targets PTEN. Other suitable oligonucleotides are described further below.

In one embodiment, gastrointestinal absorption of the composition is greater than gastrointestinal absorption of the oligonucleotide alone. In one embodiment, gastrointestinal perfusion of the composition is greater than gastrointestinal perfusion of the oligonucleotide alone. In certain embodiments, the formulation comprises one or more compounds that enhance mucosal penetration, mucosal diffusion or both mucosal penetration and diffusion.

In certain embodiments, the oil emulsion formulation further comprises at least one gastrointestinal delivery enhancer (GDE), non-limiting examples of which are described in detail in subsection II below.

In certain embodiments, the oil emulsion formulation further comprises at least one enhancer of mucosal penetration and/or diffusion. Such enhancers of mucosal penetration and/or diffusion can enhance local mucosal absorption and/or enhance systemic bioavailability of the oligonucleotide in the formulation. Non-limiting examples of enhancers of mucosal penetration and/or diffusion include sodium tartrate, calcium D-gluconate, zinc acetate, calcium phosphate amorphous nanopowder, calcium phosphate, caffeine, alpha cyclodextrin, potassium pyrophosphate, xylitol, D-mannitol, choline bitartarate, choline chloride, alginic acids and calcium citrate.

II. Gastrointestinal Delivery Enhancers

As described in the Examples, oligonucleotide formulations comprising a variety of different gastrointestinal delivery enhancers (GDE) have been found to exhibit enhanced gastrointestinal deliver of the oligonucleotide (e.g., LNA-containing gapmer), as compared to delivery of the oligonucleotide alone (i.e., in the absence of the GDE).

Accordingly, in one aspect, the disclosure provides a composition for gastrointestinal delivery, the composition comprising: (i) an oligonucleotide; and (ii) a gastrointestinal delivery enhancer (GDE) selected from the group consisting of calcium salts, potassium salts, sodium salts, ammonium salts, dicarboxylic acids, cholines, chlorides, amino sugars, fatty acids, parabens, buffering agents, clays and oils, wherein gastrointestinal delivery of the composition is greater than gastrointestinal delivery of the oligonucleotide alone.

In one embodiment, the GDE is a calcium salt. In one embodiment, the calcium salt is selected from the group consisting of calcium carbonate, calcium phosphate monobasic, calcium amorphous nanoparticles, calcium D-gluconate and alginic acid calcium. Other non-limiting examples of calcium salts include calcium acetate hydrate, calcium chloride, calcium citrate (tetrahydrate), calcium fluoride, calcium iodate, calcium L-lactate hydrate, calcium phosphate dibasic, calcium silicate and glycerol phosphate calcium salt.

In one embodiment, the GDE is a potassium salt. In one embodiment, the potassium salt is selected from the group consisting of potassium phosphate dibasic and potassium disulfide. Other non-limiting examples of potassium salts include potassium acetate, potassium bromide, potassium carbonate, potassium chloride, potassium citrate (tribasic), potassium disulfite, potassium gluconate, potassium iodate, potassium nitrate, potassium phosphate, potassium phosphate (monobasic), potassium pyrophosphate, potassium silicate and alginic acid potassium salt.

In one embodiment, the GDE is a sodium salt. In one embodiment, the sodium salt is selected from the group consisting of sodium metabisulfite, sodium azide, sodium perchlorate monohydrate and 3-(trimethylsilyl)-1-propanesulfonic acid sodium. Other non-limiting examples of sodium salts include alginic acid sodium salt, beta-glycero phosphate disodium salt, sodium acetate (trihydrate), sodium bicarbonate, sodium cacodylate (trihydrate), sodium carbonate, sodium chloride, sodium citrate (dihydrate), sodium dodecyl sulfate, sodium fluoride, sodium gluconate, sodium glycholate, sodium glycochenodeoxycholate, sodium hyaluronate, sodium hydroxide, sodium iodide, sodium malonate (dibasic), sodium nitrite, sodium perchlorate hydrate, sodium phosphate (dibasic), sodium phosphate monobasic, sodium pyrophophate tetrabasic, sodium salicylate, sodium sulfite, sodium tartrate dihydrate (dibasic), sodium taurocholate hydrate, sodium tetraborate decahydrate and sodium-L-ascorbate.

In one embodiment, the GDE is an ammonium salt. In one embodiment, the ammonium salt is ammonium iron citrate. Other non-limiting examples of ammonium salts include alginic acid ammonium salt, ammonium aluminum sulfate dodecahydrate, ammonium carbonate, ammonium chloride and ammonium molybdate.

In one embodiment, the GDE is a dicarboxylic acid. In one embodiment, the dicarboxylic acid is adipic acid. Other non-limiting examples of dicarboxylic acids include oxalic acid, malonic acid, succinic acid, glutaric acid, pimelic acid and suberic acid.

In one embodiment, the GDE is a choline. In one embodiment, the choline is choline bitartrate. Another non-limiting example of a choline is choline chloride.

In one embodiment, the GDE is a chloride. In one embodiment, the chloride is Tin (II) chloride. Other non-limiting examples of chlorides include iron (II) chloride (tetrahydrate) and zinc chloride.

In one embodiment, the GDE is an amino sugar. In one embodiment, the amino sugar is meglumine.

In one embodiment, the GDE is a fatty acid. In one embodiment, the fatty acid is octanoic acid or 4-ethyloctanoic acid.

In one embodiment, the GDE is a paraben. In one embodiment, the paraben is methylparaben or ethyl paraben.

In one embodiment, the GDE is a buffering agent. In one embodiment, the buffering agent is HEPES or Tris base.

In one embodiment, the GDE is a clay. In one embodiment, the clay is kaolin.

In one embodiment, the GDE is an oil. In one embodiment, the oil is corn oil or vegetable oil. Other non-limiting examples of oil are described above.

The oligonucleotide compositions comprising a GDE can be prepared by standard methods known in the art, such as described in the Examples.

In one embodiment, the oligonucleotide is an antisense oligonucleotide (e.g., antisense RNA). In one embodiment, the antisense oligonucleotide comprises at least one locked nucleic acid (LNA), referred to herein as an LNA oligonucleotide. In one embodiment, the LNA oligonucleotide targets HIF-1 alpha. In one embodiment, the LNA oligonucleotide targets PTEN. Other suitable oligonucleotides are described further below.

In one embodiment, gastrointestinal absorption of the composition is greater than gastrointestinal absorption of the oligonucleotide alone. In one embodiment, gastrointestinal perfusion of the composition is greater than gastrointestinal perfusion of the oligonucleotide alone. In certain embodiments, the formulation comprises one or more compounds that enhance mucosal penetration, mucosal diffusion or both mucosal penetration and diffusion.

In certain embodiments, the GDE-containing formulation further comprises an oil emulsion, non-limiting examples of which are described in detail in subsection I above.

In certain embodiments, the GDE-containing formulation further comprises at least one enhancer of mucosal penetration and/or diffusion. Such enhancers of mucosal penetration and/or diffusion can enhance local mucosal absorption and/or enhance systemic bioavailability of the oligonucleotide in the formulation. Non-limiting examples of enhancers of mucosal penetration and/or diffusion include sodium tartrate, calcium D-gluconate, zinc acetate, calcium phosphate amorphous nanopowder, calcium phosphate, caffeine, alpha cyclodextrin, potassium pyrophosphate, xylitol, D-mannitol, choline bitartarate, choline chloride, alginic acids and calcium citrate.

III. Gastrointestinal Perfusion and/or Absorption Enhancers for Specific LNAs

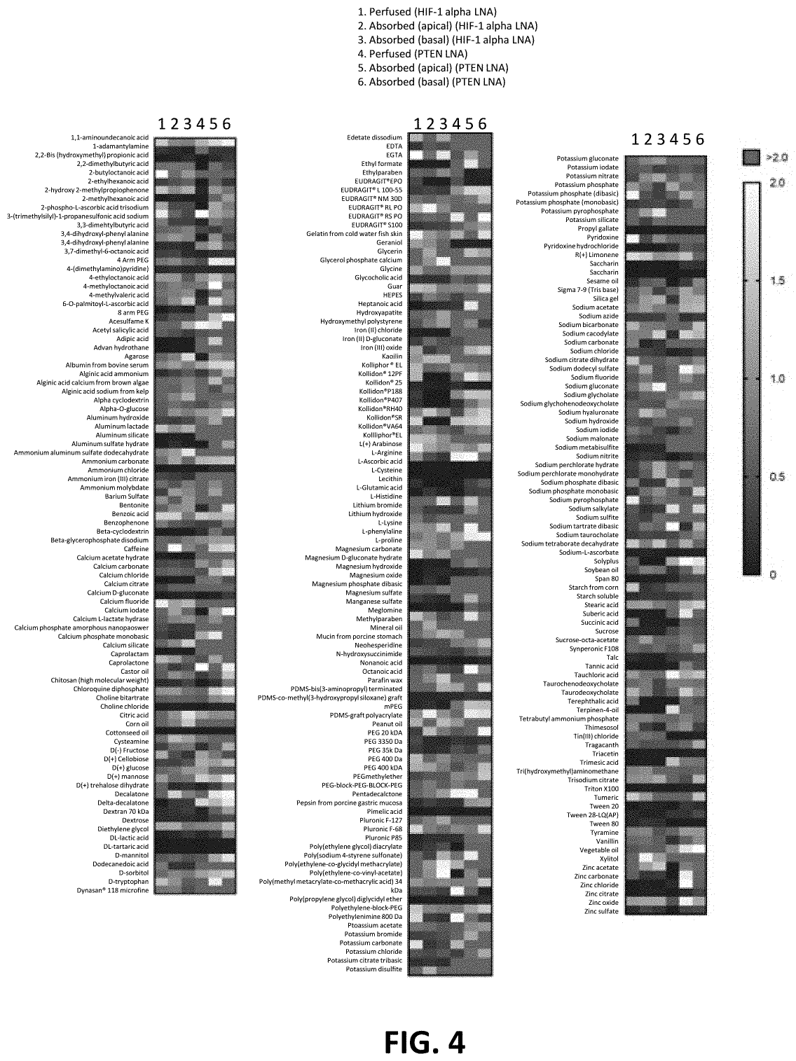

As described in Example 3, a large diverse chemical compound library, containing compounds representing a wide range of chemical properties, was screened to identify compounds that enhanced gastrointestinal absorption and/or perfusion of a LNA specific for either HIF-1 alpha or PTEN. As used herein, the term gastrointestinal “absorption” refers to modulation of local intestinal tissue uptake for topical treatment. As used herein, the term gastrointestinal “perfusion” refers to modulation of permeation through the gastrointestinal tissue (e.g., for potential enhanced systemtic bioavailability). As demonstrated in the data shown in FIG. 4, different panels of compounds were identified that enhanced the perfusion and/or absorption of the HIF-1 alpha LNA or the PTEN LNA, although there was some overlap in the identified compounds.

Based on the screening of the chemical library (as described in Example 3), compounds were identified that enhanced the gastrointestinal perfusion or absorption enhancer of the HIF-1 alpha LNA. Accordingly, in one aspect, the disclosure pertains to a composition for gastrointestinal delivery, the composition comprising:
(a) a locked nucleic acid oligonucleotide that targets HIF-1 alpha; and
(b) a gastrointestinal perfusion or absorption enhancer selected from the group consisting of vegetable oil, 3-(Trimethylsilyl)-propanesulfonic acid sodium, 4-methyloctanoic acid; 8 arm PEG, advan hydrothane, alginic acid ammonium, alginic acid calcium, alginic acid potassium, benzophenone, beta-alanine, calcium D-gluconate, calcium phosphate amorphous nanopowder, calcium silicate, choline bitartarate, choline chloride, D(+) cellobiose, D(+) Trehalose dihydrate, ethylparaben, glycerin, glycerol phosphate calcium, hydroxyapatite, L-histidine, magnesium phosphate dibasic, methyl paraben, octanoic acid, paraffin wax, pentadecalactone, Pluronic® F-127, Poly(sodium) 4-styrene sulfonate, Poly(ethylene-co-glycidyl methacrylate), Poly(ethylene-co-vinyl-acetate), potassium disulfite, potassium gluconate, potassium phosphate dibasic, potassium pyrophosphate, potassium silicate, Sigma 7-9 (Tris base), silica gel, sodium dodecyl sulfate, sodium gluconate, sodium hyaluronate, Tin (II) chloride, xylitol, zinc acetate, 8 arm PEG, calcium D-gluconate, calcium phosphate monobasic, Koliphor® EL, paraffin wax, peanut oil, PEG 400 Da, potassium disulfite, sodium perchlorate monohydrate, sodium tartrate dibasic, sucrose octa-acetate, Tin (II) chloride and Tris (hydroxymethyl) aminomethane.

In certain embodiments of the HIF-1 alpha LNA composition, the gastrointestinal perfusion or absorption enhancer is selected from the group consisting of vegetable oil, calcium phosphate amorphous nanopowder, choline bitartarate, calcium phosphate monobasic, Tin (II) chloride, methylparaben, calcium D-gluconate, potassium disulfite, sodium perchlorate monohydrate, alginic acid calcium, Sigma 7-9 (Tris base), ethyl paraben, 3-(Trimethylsilyl)-1-propanesulfonic acid sodium and potassium phosphate dibasic.

In certain embodiments of the HIF-1 alpha LNA composition, the gastrointestinal perfusion or absorption enhancer is selected from the group consisting of calcium phosphate monobasic, Tin (II) chloride, methylparaben, calcium D-gluconate, potassium disulfite.

In certain embodiments of the HIF-1 alpha LNA composition, the gastrointestinal perfusion or absorption enhancer is selected from the group consisting of vegetable oil, calcium phosphate amorphous nanopowder and choline bitartarate.

In certain embodiments of the HIF-1 alpha LNA composition, the gastrointestinal perfusion enhancer is selected from the group consisting of 3-(Trimethylsilyl)-propanesulfonic acid sodium, 4-methyloctanoic acid; 8 arm PEG, advan hydrothane, alginic acid ammonium, alginic acid calcium, alginic acid potassium, benzophenone, beta-alanine, calcium D-gluconate, calcium phosphate amorphous nanopowder, calcium silicate, choline bitartarate, choline chloride, D(+) cellobiose, D(+) Trehalose dihydrate, ethylparaben, glycerin, glycerol phosphate calcium, hydroxyapatite, L-histidine, magnesium phosphate dibasic, methyl paraben, octanoic acid, paraffin wax, pentadecalactone, Pluronic® F-127, Poly(sodium) 4-styrene sulfonate, Poly(ethylene-co-glycidyl methacrylate), Poly(ethylene-co-vinyl-acetate), potassium disulfite, potassium gluconate, potassium phosphate dibasic, potassium pyrophosphate, potassium silicate, Sigma 7-9 (Tris base), silica gel, sodium dodecyl sulfate, sodium gluconate, sodium hyaluronate, Tin (II) chloride, xylitol and zinc acetate.

In certain embodiments of the HIF-1 alpha LNA composition, the gastrointestinal perfusion enhancer is selected from the group consisting of alginic acid calcium, Sigma 7-9 (Tris base), ethyl paraben, 3-(Trimethylsilyl)-1-propanesulfonic acid sodium and potassium phosphate dibasic.

In certain embodiments of the HIF-1 alpha LNA composition, the gastrointestinal absorption enhancer is selected from the group consisting of 8 arm PEG, calcium D-gluconate, calcium phosphate monobasic, Koliphor® EL, paraffin wax, peanut oil, PEG 400 Da, potassium disulfite, sodium perchlorate monohydrate, sodium tartrate dibasic, sucrose octa-acetate, Tin (II) chloride and Tris (hydroxymethyl) aminomethane. In certain embodiments, the gastrointestinal absorption enhancer is sodium perchlorate monohydrate.

Also based on the screening of the chemical library (as described in Example 3), compounds were identified that enhanced the gastrointestinal perfusion or absorption enhancer of the PTEN LNA. Accordingly, in another aspect, the disclosure pertains to a composition for gastrointestinal delivery, the composition comprising:
(a) a locked nucleic acid oligonucleotide that targets PTEN; and
(b) a gastrointestinal perfusion or absorption enhancer selected from the group consisting of 2-butyloctanoic acid 4-methyl valeric acid, acetyl salicylic acid, adipic acid, alginic acid ammonium, alginic acid potassium, alpha D-glucose, aluminum hydroxide, aluminum oxide, ammonium aluminum sulfate dodecahydrate, ammonium carbonate, ammonium chloride, ammonium iron (III) citrate, beta-alanine, beta-cyclodextrin, calcium carbonate, calcium citrate, calcium fluoride, calcium iodate, calcium phosphate amorphous nanopowder, calcium phosphate dibasic, choline chloride, D(+) Trehalose dihydrate, corn oil, dodecanedoic acid, D-tryptophan, Dynasan® 118 microfine, edatate disodium, EDTA, ethyl formate, ethylparaben, EUDRAGIT® RL PO, glycerin, glycerol phosphate calcium, hydroxyapatite, hydroxymethyl polystyrene, Iron (III) chloride, Iron (III) oxide, Kaolin, Kollidon® 12PF, Kolliphor® EL, L-histidine, lithium hydroxide, magnesium carbonate, magnesium oxide, magnesium phosphate dibasic, magnesium sulfate, Meglumine, methyl paraben, PEG 400 Da, Pluronic® F-127, potassium bromide, potassium citrate tribasic, potassium disulfite, potassium gluconate, potassium nitrate, potassium phosphate (dibasic), potassium pyrophosphate, potassium silicate, pyridoxine, Sigma 7-9 (Tris base), sodium azide, sodium bicarbonate, sodium carbonate, sodium dodecyl sulfate, sodium fluoride, sodium gluconate, sodium hyaluronate, sodium hydroxide, sodium malonate, sodium metabisulfite, sodium perchlorate hydrate, sodium perchlorate monohydrate, sodium phosphate monobasic, sodium pyrophosphate, sodium sulfite, sodium tetraborate decahydrate, starch from corn, suberic acid, sucrose octa-acetate, Taurodeoxycholate, Tetrabutyl ammonium phosphate, Tris (hydroxymethyl) aminomethane, Trisodium citrate, turmeric, xylitol, 2-butyloctanoic acid, 2-hydroxy 2-methyl propiophenone, 3,4-dihydroxyl 1-phenyl alanine, 4-ethyloctanoic acid, 4-methylnonanoic acid, 4-methylvaleric acid, 8 arm PEG, alpha cyclodextrin, aluminum lactate, ammonium molybdate, calcium L-lactate hydrate, calcium phosphate monobasic, calcium silicate, D(+) cellobiose, EUDRAGIT® RS PO, gelatin from cold water fish skin, HEPES, Iron (II) D-gluconate, L-lysine, L-proline, manganese sulfate, mineral oil, octanoic acid, paraffin wax, peanut oil, PEG 20 kDa, PEG-block-PEG-block-PEG, pentadecalactone, Poly(ethylene glycol) diacrylate, Poly(sodium 4-styrene sulfonate), Poly(ethylene-co-glycidyl methacrylate), Poly(propyl glycol) diglycidyl ether, potassium carbonate, R(+)-Limonene, sodium salicylate, Terpin-4-ol and zinc carbonate.

In certain embodiments of the PTEN LNA composition, the gastrointestinal perfusion or absorption enhancer is selected from the group consisting of calcium carbonate, adipic acid, Kaolin, ammonium iron citrate, sodium metabisulfite, HEPES, corn oil, 4-ethyloctanoic acid, calcium phosphate monobasic, octanoic acid, sodium azide, sodium perchlorate monohydrate, potassium phosphate (dibasic), Sigma 7-9 (Tris base) and Meglumine.

In certain embodiments of the PTEN LNA composition, the gastrointestinal perfusion or absorption enhancer is selected from the group consisting of calcium carbonate, adipic acid, Kaolin, ammonium iron citrate and sodium metabisulfite.

In certain embodiments of the PTEN LNA composition, the gastrointestinal perfusion enhancer is selected from the group consisting of 2-butyloctanoic acid 4-methyl valeric acid, acetyl salicylic acid, adipic acid, alginic acid ammonium, alginic acid potassium, alpha D-glucose, aluminum hydroxide, aluminum oxide, ammonium aluminum sulfate dodecahydrate, ammonium carbonate, ammonium chloride, ammonium iron (III) citrate, beta-alanine, beta-cyclodextrin, calcium carbonate, calcium citrate, calcium fluoride, calcium iodate, calcium phosphate amorphous nanopowder, calcium phosphate dibasic, choline chloride, D(+) Trehalose dihydrate, dodecanedoic acid, D-tryptophan, Dynasan® 118 microfine, edatate disodium, EDTA, ethyl formate, ethylparaben, EUDRAGIT® RL PO, glycerin, glycerol phosphate calcium, hydroxyapatite, hydroxymethyl polystyrene, Iron (III) chloride, Iron (III) oxide, Kaolin, Kollidon® 12PF, Kolliphor® EL, L-histidine, lithium hydroxide, magnesium carbonate, magnesium oxide, magnesium phosphate dibasic, magnesium sulfate, Meglumine, methyl paraben, PEG 400 Da, Pluronic® F-127, potassium bromide, potassium citrate tribasic, potassium disulfite, potassium gluconate, potassium nitrate, potassium phosphate (dibasic), potassium pyrophosphate, potassium silicate, pyridoxine, Sigma 7-9 (Tris base), sodium azide, sodium bicarbonate, sodium carbonate, sodium dodecyl sulfate, sodium fluoride, sodium gluconate, sodium hyaluronate, sodium hydroxide, sodium malonate, sodium metabisulfite, sodium perchlorate hydrate, sodium perchlorate monohydrate, sodium phosphate monobasic, sodium pyrophosphate, sodium sulfite, sodium tetraborate decahydrate, starch from corn, suberic acid, sucrose octa-acetate, Taurodeoxycholate, Tetrabutyl ammonium phosphate, Tris (hydroxymethyl) aminomethane, Trisodium citrate, turmeric and xylitol.

In certain embodiments of the PTEN LNA composition, the gastrointestinal perfusion enhancer is selected from the group consisting of sodium azide, sodium perchlorate monohydrate, potassium phosphate (dibasic), Sigma 7-9 (Tris base) and Meglumine.

In certain embodiments of the PTEN LNA composition, the gastrointestinal absorption enhancer is selected from the group consisting of 2-butyloctanoic acid, 2-hydroxy 2-methyl propiophenone, 3,4-dihydroxyl 1-phenyl alanine, 4-ethyloctanoic acid, 4-methylnonanoic acid, 4-methylvaleric acid, 8 arm PEG, acetyl salicylic acid, adipic acid, alginic acid ammonium, alginic acid potassium, alpha cyclodextrin, alpha D-glucose, aluminum hydroxide, aluminum lactate, aluminum oxide, ammonium aluminum sulfate dodecahydrate, ammonium carbonate, ammonium chloride, ammonium iron (III) citrate, ammonium molybdate, beta-cyclodextrin, calcium carbonate, calcium citrate, calcium fluoride, calcium iodate, calcium L-lactate hydrate, calcium phosphate amorphous nanopowder, calcium phosphate dibasic, calcium phosphate monobasic, calcium silicate, choline chloride, D(+) cellobiose, corn oil, dodecanedoic acid, D-tryptophan, Dynasan® 118 microfine, edatate disodium, EDTA, ethyl formate, EUDRAGIT® RL PO, EUDRAGIT® RS PO, gelatin from cold water fish skin, glycerol phosphate calcium, HEPES, hydroxyapatite, Iron (III) chloride, Iron (II) D-gluconate, Kaolin, Kolliphor® EL, L-histidine, lithium hydroxide, L-lysine, L-proline, magnesium carbonate, magnesium oxide, magnesium phosphate dibasic, magnesium sulfate, manganese sulfate, methyl paraben, mineral oil, octanoic acid, paraffin wax, peanut oil, PEG 20 kDa, PEG 400 Da, PEG-block-PEG-block-PEG, pentadecalactone, Pluronic® F-127, Poly(ethylene glycol) diacrylate, Poly(sodium 4-styrene sulfonate), Poly(ethylene-co-glycidyl methacrylate), Poly(propyl glycol) diglycidyl ether, potassium bromide, potassium carbonate, potassium citrate tribasic, potassium disulfite, potassium gluconate, potassium nitrate, potassium pyrophosphate, potassium silicate, pyridoxine, R(+)-Limonene, sodium bicarbonate, sodium carbonate, sodium fluoride, sodium gluconate, sodium hyaluronate, sodium hydroxide, sodium malonate, sodium metabisulfite, sodium perchlorate hydrate, sodium pyrophosphate, sodium salicylate, sodium sulfite, sodium tetraborate decahydrate, starch from corn, suberic acid, sucrose octa-acetate, Terpin-4-ol, Tetrabutyl ammonium phosphate, Tris (hydroxymethyl) aminomethane, turmeric, xylitol and zinc carbonate.

In certain embodiments of the PTEN LNA composition, the gastrointestinal absorption enhancer is selected from the group consisting of HEPES, corn oil, 4-ethyloctanoic acid, calcium phosphate monobasic and octanoic acid.

IV. Oil Emulsions as Gastrointestinal Delivery Enhancers for Specific LNAs

As further described in Example 3, since the initial screen of the chemical library indicated that LNA oil emulsions exhibited enhanced tissue perfusion and absorption properties, another screen was performed using a large panel of organic oils combined with different emulsifiers (the commercially available Soluplus®, Pluronic® F127 and Tween® 20 emulsifiers). The oils and emulsifiers are combined through a standard dispersion process (as described in the examples) to prepare the oil emulsion.

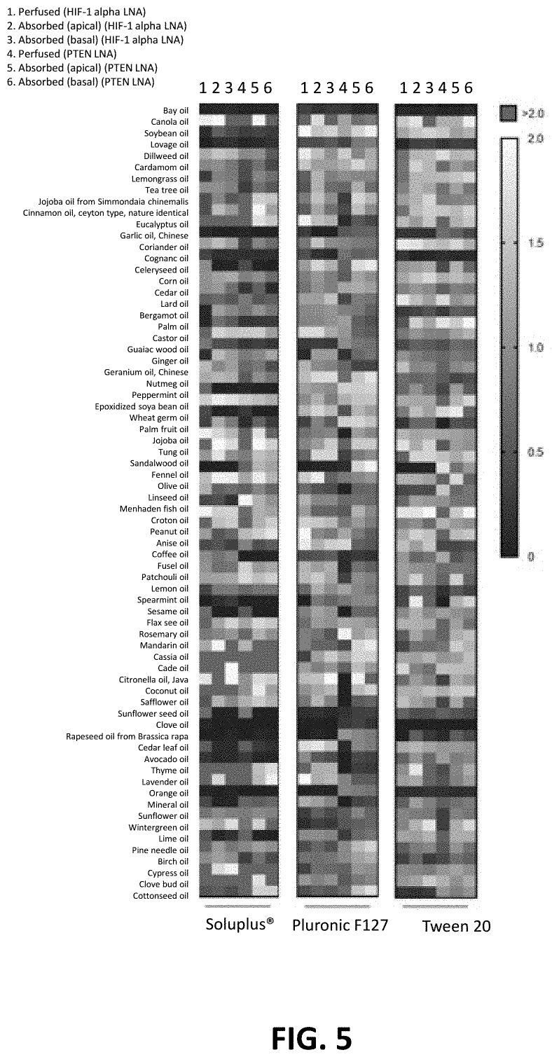

Based on the screening of the panel of oil emulsions (as described in Example 3 and FIG. 5), oil emulsions were identified that enhanced the gastrointestinal perfusion or absorption enhancer of the HIF-1 alpha LNA. Accordingly, in another aspect, the disclosure pertains to a composition for gastrointestinal delivery, the composition comprising:
(a) a locked nucleic acid oligonucleotide that targets HIF-1 alpha; and
(b) a gastrointestinal perfusion or absorption enhancer comprising an oil emulsion selected from the group consisting of:
   (i) Soluplus® emulsified with an oil selected from the group consisting of canola oil, Eucalyptus oil, castor oil, tung oil, mandarin oil, peanut oil, flax seed oil, Cassia oil, cade oil, citronella oil, coconut oil, thyme oil, lavender oil, cypress oil and clove bud oil; or
   (ii) Pluronic® F-127 emulsified with an oil selected from the group consisting of canola oil, olive oil, sandalwood oil, croton oil, mandarin oil and thyme oil; or
   (iii) Tween® 20 emulsified with an oil selected from the group consisting of sandalwood oil, canola oil, vegetable oil, thyme oil and lavender oil.

In one embodiment, the HIF-1 alpha LNA composition comprises an oil emulsion that enhances gastrointestinal perfusion selected from the group consisting of: (i) Soluplus® emulsified with an oil selected from the group consisting of Eucalyptus oil, castor oil, tung oil, peanut oil, flax seed oil, Cassia oil, cade oil, coconut oil, thyme oil, lavender oil, cypress oil and clove bud oil; or (ii) Pluronic® F-127 emulsified with an oil selected from the group consisting of canola oil, sandalwood oil, croton oil, mandarin oil and thyme oil; or (iii) Tween® 20 emulsified with sandalwood oil.

In another embodiment, the HIF-1 alpha LNA composition comprises an oil emulsion that enhances gastrointestinal absorption selected from the group consisting of: (i) Soluplus® emulsified with an oil selected from the group consisting of canola oil, Eucalyptus oil, mandarin oil, Cassia oil, cade oil, citronella oil, coconut oil, thyme oil, lavender oil and clove bud oil; or (ii) Pluronic® F-127 emulsified with an oil selected from the group consisting of canola oil, olive oil, croton oil and mandarin oil; or (iii) Tween® 20 emulsified with an oil selected from the group consisting of canola oil, vegetable oil, thyme oil and lavender oil.

Also based on the screening of the panel of oil emulsions (as described in Example 3 and FIG. 5), oil emulsions were identified that enhanced the gastrointestinal perfusion or absorption enhancer of the PTEN alpha LNA. Accordingly, in another aspect, the disclosure pertains to a composition for gastrointestinal delivery, the composition comprising:
(a) a locked nucleic acid oligonucleotide that targets PTEN; and
(b) a gastrointestinal perfusion or absorption enhancer comprising an oil emulsion selected from the group consisting of:
   (i) Soluplus® emulsified with an oil selected from the group consisting of canola oil, jojoba oil, cinnamon oil, Eucalyptus oil, tung oil, fennel oil, peanut oil, Cassia oil, cade oil, thyme oil, lavender oil, mineral oil, mandarin oil, wintergreen oil, cypress oil, clove bud oil and cottonseed oil; or
   (ii) Pluronic® F-127 emulsified with an oil selected from the group consisting of celery seed oil, tung oil, citronella oil and cade oil; or
   (iii) Tween® 20 emulsified with an oil selected from the group consisting of Eucalyptus oil, geranium oil, epoxidized soya bean oil, olive oil, croton oil, anise oil, lemon oil, flax seed oil, wheat germ oil and rosemary oil.

In one embodiment, the PTEN LNA composition comprises an oil emulsion that enhances gastrointestinal perfusion selected from the group consisting of: (i) Soluplus® emulsified with an oil selected from the group consisting of canola oil, jojoba oil, cinnamon oil, Eucalyptus oil, tung oil, fennel oil, peanut oil, Cassia oil, cade oil, thyme oil, lavender oil, mineral oil, cypress oil, clove bud oil and cottonseed oil; or (ii) Pluronic® F-127 emulsified with an oil selected from the group consisting of celery seed oil, tung oil and cade oil; or (iii) Tween® 20 emulsified with an oil selected from the group consisting of Eucalyptus oil, geranium oil, epoxidized soya bean oil, olive oil, croton oil, anise oil, lemon oil, flax seed oil and rosemary oil.

In another embodiment, the PTEN LNA composition comprises an oil emulsion that enhances gastrointestinal absorption selected from the group consisting of: (i) Soluplus® emulsified with an oil selected from the group consisting of canola oil, jojoba oil, mandarin oil, Cassia oil, cade oil, wintergreen oil, cypress oil and clove bud oil; or (ii) Pluronic® F-127 emulsified with citronella oil; or (iii) Tween® 20 emulsified with an oil selected from the group consisting of wheat germ oil, olive oil and lemon oil.

V. Mucosal Penetration and/or Diffusion Enhancers

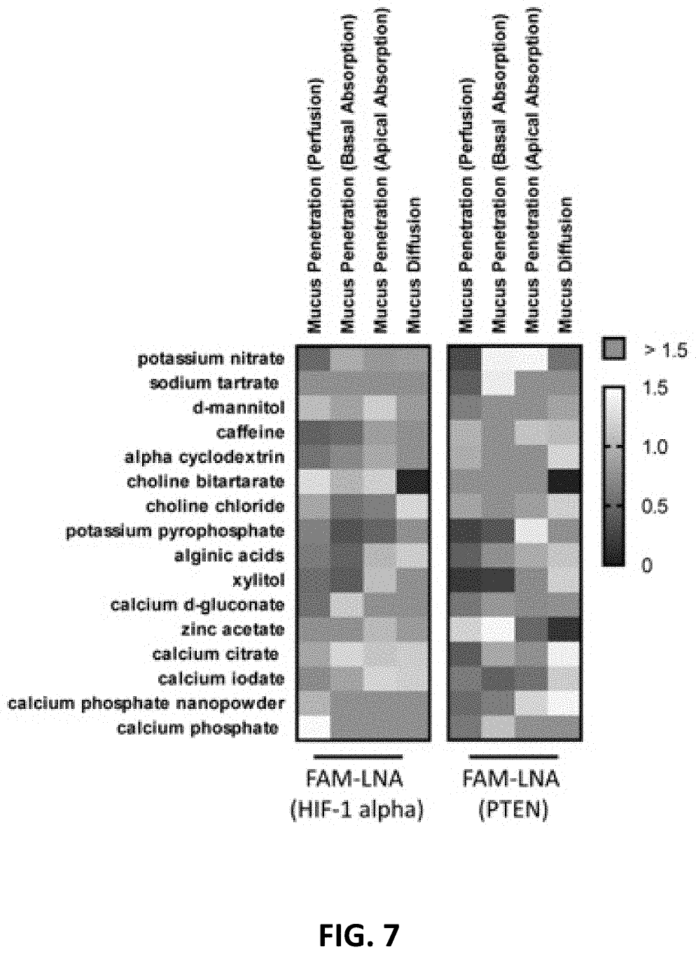

As described in Example 4, a subpanel of compounds identified from prior screens were studied for their ability to enhance mucosal penetration and/or diffusion. As demonstrated in the data shown in FIG. 7, panels of compounds were identified that enhanced the mucosal penetration and/or diffusion of the HIF-1 alpha LNA or the PTEN LNA.

Accordingly, in one aspect, the disclosure pertains to a composition for gastrointestinal delivery, the composition comprising:
(a) a locked nucleic acid oligonucleotide that targets HIF-1 alpha; and
(b) a gastrointestinal mucus penetration or diffusion enhancer selected from the group consisting of sodium tartrate, calcium D-gluconate, zinc acetate, calcium phosphate amorphous nanopowder, calcium phosphate, caffeine, alpha cyclodextrin, potassium pyrophosphate and xylitol.

In certain embodiments of the HIF-1 alpha LNA composition, the gastrointestinal mucus penetration enhancer is selected from the group consisting of sodium tartrate, calcium D-gluconate, zinc acetate, calcium phosphate amorphous nanopowder and calcium phosphate.

In certain embodiments of the HIF-1 alpha LNA composition, the gastrointestinal mucus diffusion enhancer is selected from the group consisting of sodium tartrate, caffeine, alpha cyclodextrin, potassium pyrophosphate, xylitol, calcium D-gluconate, calcium phosphate amorphous nanopowder and calcium phosphate.

In yet another aspect, the disclosure pertains to a composition for gastrointestinal delivery, the composition comprising:
(a) a locked nucleic acid oligonucleotide that targets PTEN; and
(b) a gastrointestinal mucus penetration or diffusion enhancer selected from the group consisting of sodium tartrate, D-mannitol, caffeine, alpha cyclodextrin, choline bitartarate, choline chloride, alginic acids, calcium citrate, calcium phosphate, potassium pyrophosphate and calcium D-gluconate.

In certain embodiments of the PTEN LNA composition, the gastrointestinal mucus penetration enhancer is selected from the group consisting of sodium tartrate, D-mannitol, caffeine, alpha cyclodextrin, choline bitartarate, choline chloride, alginic acids, calcium citrate and calcium phosphate.

In certain embodiments of the PTEN LNA composition, the gastrointestinal mucus diffusion enhancer is selected from the group consisting of sodium tartrate, potassium pyrophosphate, calcium D-gluconate and calcium phosphate.

VI. Additional Compositions for Enhanced Gastrointestinal Delivery

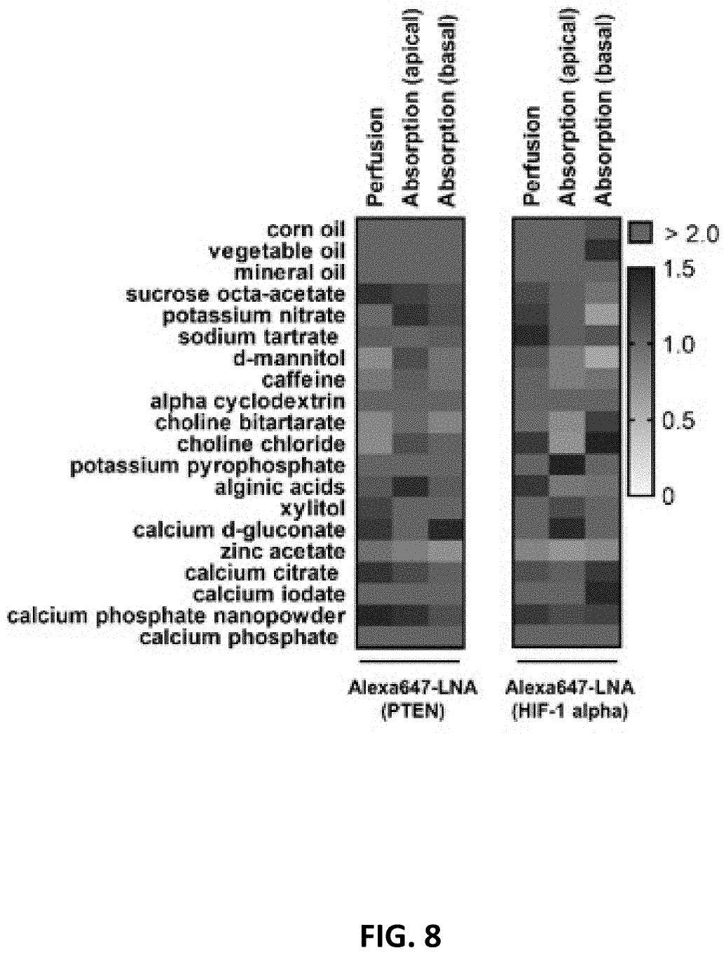

Further in vitro and in vivo analyses were conducted on certain selected formulations, as described in Examples 5 and 6. As demonstrated in the data shown in FIGS. 8 and 10, additional subpanels of compounds were identified that enhanced the gastrointestinal absorption and/or perfusion of the HIF-1 alpha LNA or the PTEN LNA.

Accordingly, in yet another aspect, the disclosure pertains to a composition for gastrointestinal delivery, the composition comprising:
(a) a locked nucleic acid oligonucleotide that targets HIF-1 alpha; and
(b) a gastrointestinal perfusion or absorption enhancer selected from the group consisting of corn oil, vegetable oil, mineral oil, alpha cyclodextrin, potassium pyrophosphate, xylitol, calcium D-gluconate, calcium iodate, calcium phosphate, calcium citrate tetrahydrate, sodium glycholate, an oil emulsion comprising celery oil and Pluronic® F-127, D-mannitol, caffeine, choline chloride, potassium pyrophosphate, calcium phosphate dibasic, methyl paraben, an oil emulsion comprising clove bud oil and Soluplus® and an oil emulsion comprising lemon oil and Tween® 20.

In yet another aspect, the disclosure pertains to a composition for gastrointestinal delivery, the composition comprising:
(a) a locked nucleic acid oligonucleotide that targets PTEN; and
(b) a gastrointestinal perfusion or absorption enhancer selected from the group consisting of corn oil, vegetable oil, mineral oil, alpha cyclodextrin, potassium pyrophosphate, calcium iodate, calcium phosphate, sodium tartrate, xylitol, calcium D-gluconate, D-mannitol, sodium glycholate and an oil emulsion comprising celery oil and Pluronic® F-127.

While the HIF-1 alpha LNA formulations and PTEN LNA formulations described herein in Subsection I-IV have been described using Markus groups of compounds, all formulations comprising an LNA of the disclosure (HIF-1 alpha or PTEN) and any single one of the compounds listed with a Markus group as disclosed herein are also contemplated by the invention and intended to be encompassed by the disclosure.

VII. Oligonucleotides

As used herein, the term “oligonucleotide” includes RNA agents and DNA agents, as well as chimeric oligonucleotides that comprise both RNA and DNA elements (e.g., gapmers). Moreover, the term “oligonucleotide” includes compounds comprising naturally-occurring nucleotides, non-naturally-occurring nucleotides (e.g., nucleotide analogues) or a combination of naturally-occurring and non-naturally-occurring nucleotides. In one embodiment, the oligonucleotide is an RNA agent (i.e., an oligonucleotide whose sugar-phosphate backbone comprises ribose, or a chemical analogue thereof). In one embodiment, the oligonucleotide is a DNA agent (i.e., an oligonucleotide whose sugar-phosphate backbone comprises deoxyribose, or a chemical analogue thereof). In one embodiment, the oligonucleotide is a modified RNA agent, a non-limiting example of which is a locked nucleic acid (LNA)-containing RNA oligonucleotide (described further below).

RNA agents include single-stranded RNA, double-stranded RNA (dsRNA) or a molecule that is a partially double-stranded RNA, i.e., has a portion that is double-stranded and a portion that is single-stranded. The RNA molecule can be a circular RNA molecule or a linear RNA molecule. Such oligonucleotides are well established in the art.

DNA agents include double-stranded DNA, single-stranded DNA (ssDNA), or a molecule that is a partially double-stranded DNA, i.e., has a portion that is double-stranded and a portion that is single-stranded. In some cases the DNA molecule is triple-stranded or is partially triple-stranded, i.e., has a portion that is triple stranded and a portion that is double stranded. The DNA molecule can be a circular DNA molecule or a linear DNA molecule. Such oligonucleotides are well established in the art.

Non-limiting examples of RNA agents include messenger RNAs (mRNAs) (e.g., encoding a protein of interest), modified mRNAs (mmRNAs) that include at least one chemical modification as compared to naturally-occurring RNA, mRNAs that incorporate a micro-RNA binding site(s) (miR binding site(s)), modified RNAs that comprise functional RNA elements, microRNAs (miRNAs), antagomirs, small (short) interfering RNAs (siRNAs) (including shortmers and dicer-substrate RNAs), RNA interference (RNAi) molecules, antisense RNAs, ribozymes, small hairpin RNAs (shRNA) and locked nucleic acids (LNAs). Such RNA agents are well established in the art.

In one embodiment, the oligonucleotide is an antisense oligonucleotide, e.g., an antisense RNA. Antisense RNAs (asRNAs), also referred to in the art as antisense transcripts, are naturally-occurring or synthetically produced single-stranded RNA molecules that are complementary to a protein-coding messenger RNA (mRNA) with which it hybridizes and thereby blocks the translation of the mRNA into a protein. Antisense transcript are classified into short (less than 200 nucleotides) and long (greater than 200 nucleotides) non-coding RNAs (ncRNAs). The primary natural function of asRNAs is in regulating gene expression and synthetic versions have been used widely as research tools for gene knockdown and for therapeutic applications. Antisense RNAs and their functions have been described in the art (see e.g., Weiss et al. (1999) Cell. Molec. Life Sci. 55:334-358; Wahlstedt (2013) Nat. Rev. Drug Disc. 12:433-446; Pelechano and Steinmetz (2013) Nat. Rev. Genet. 14:880-893). Accordingly, in one embodiment, a formulation of the disclosure comprises an agent for antisense therapy. In one embodiment, the agent for antisense therapy is an RNA agent or chimeric oligonucleotide (e.g., gapmer) comprising at least one modification as compared to naturally-occurring ribonucleic acids, such as at least one chemical analogue of a naturally-occurring ribonucleic acid. In one embodiment, the modification of the RNA agent, as compared to naturally-occurring ribonucleic acids, comprises incorporation of at least one locked nucleic acid.

In one embodiment, the oligonucleotide comprises one or more locked nucleic acids. Locked nucleic acids, also referred to as inaccessible RNA, are modified RNA nucleotide molecules in which the ribose moiety of the LNA is modified with an extra bridge connecting the 2′ oxygen and the 4′ carbon. This bridge “locks” the ribose in the 3′-endo (North) conformation. LNA nucleotides can be mixed with DNA or RNA residues in an oligonucleotide whenever desired and hybridize with DNA or RNA according to Watson-Crick base-pairing rules. The locked ribose conformation enhances base stacking and backbone pre-organization. This significantly increases the hybridization properties (e.g., melting temperature) of oligonucleotides containing LNA nucleotides. LNA molecules, and their properties, have been described in the art (see e.g., Obika et al. (1997) Tetrahedron Lett. 38:8735-8738; Koshkin et al. (1998) Tetrahedron 54:3607-3630; Elmen et al. (2005) Nucl. Acids Res. 33:439-447).

In one embodiment, the antisense RNA is a gapmer. Gapmers are chimeric antisense oligonucleotides that contain a central block of deoxynucleotide monomers sufficiently long to induce RNAase H cleavage. Such gapmers are well established in the art. In one embodiment, the gapmer is a locked nucleic acid (LNA)-containing gapmer. The use of LNA-containing gapmer antisense oligonucleotides for antisense therapy is well established in the art (see e.g., Wahlestedt et al. (2000) Proc. Natl. Acad. Sci. USA 97:5633-5638; Kurreck et al. (2002) Nucl. Acids Res. 30:1911-1918; Fluiter et al. (2009) Mol. Biosyst. 5:838-843; Pendergraff et al. (2017) Mol. Therap. Nucl. Acids 8:158-168).

In one embodiment, the oligonucleotide is an LNA-containing gapmer oligonucleotide that targets HIF-1 alpha. The sequence of a non-limiting example of such a gapmer is shown in SEQ ID NO: 1.

In one embodiment, the oligonucleotide is an LNA-containing gapmer oligonucleotide that targets PTEN. Sequence of a non-limiting example of such gapmers are shown in SEQ ID NOs: 3 and 4.

VIII. Preparation of Formulations

The formulations of the invention are prepared using standard preparation techniques known in the art. Oligonucleotides, such as HIF-1 alpha LNAs or PTEN LNAs, can be prepared as described in the examples (e.g., Materials and Methods description and Example 1). In certain embodiments of the HIF-1 alpha LNA compositions of the disclosure, the locked nucleic acid oligonucleotide that targets HIF-1 alpha comprises the nucleotide sequence shown in SEQ ID NO: 1. In certain embodiments of the PTEN LNA compositions of the disclosure, the locked nucleic acid oligonucleotide that targets PTEN comprises the nucleotide sequence shown in SEQ ID NO: 3 or 4.

Compounds to be combined with the oligonucleotide, e.g., LNA, to prepare a formulation of the disclosure are commercially available. Formulations can be prepared by standard methods (e.g., as described in the Materials and Methods in the Examples). For example, an aqueous oligonucleotide preparation (e.g., LNA in buffer, such as PBS) can be combined with the excipient (e.g., gastrointestinal perfusion and/or absorption enhancer) and the mixture can be mixed by pipetting (e.g., automated pipetting). For oil emulsions, an aqueous oligonucleotide preparation (e.g., LNA in buffer, such as PBS) can be combined with the oil emulsion solution and the entire mixture can be mixed by pipetting (e.g., 60 times using a liquid handling system) to generate an oil-water emulsion.

In one embodiment, an oligonucleotide formulation of the disclosure can be applied topically to gastrointestinal tissue. In another embodiment, an oligonucleotide formulation of the disclosure can be administered orally to thereby deliver it to gastrointestinal tissue. In yet another embodiment, an oligonucleotide formulation of the disclosure can be administered rectally to thereby deliver it to gastrointestinal tissue.

IX. Methods of Enhanced Delivery to Gastrointestinal Tissue

In another aspect, the disclosure provides methods of enhancing delivery of oligonucleotides to gastrointestinal tissue. Accordingly, in one aspect, the disclosure provide a method of enhancing delivery of an oligonucleotide to gastrointestinal tissue, the method comprising administering a composition of the disclosure to the gastrointestinal tissue (e.g., topically, orally, rectally).

In another embodiment, the disclosure pertains to a method of enhancing delivery of a locked nucleic acid oligonucleotide that targets HIF-1 alpha to gastrointestinal tissue, the method comprising administering any of the HIF-1 alpha LNA-containing compositions of the disclosure to the gastrointestinal tissue. In another embodiment, the disclosure pertains to a method of enhancing delivery of a locked nucleic acid oligonucleotide that targets PTEN to gastrointestinal tissue, the method comprising administering any one the PTEN LNA-containing compositions of the disclosure to the gastrointestinal tissue.

In one embodiment, the LNA-containing composition of the disclosure is administered to the gastrointestinal tissue topically. In one embodiment, the LNA-containing composition of the disclosure is administered to the gastrointestinal tissue orally. In one embodiment, the LNA-containing composition of the disclosure is administered to the gastrointestinal tissue rectally.

The compositions of the disclosure for gastrointestinal delivery can be used in a wide variety of clinical conditions pertaining to gastrointestinal-related disorders and diseases, non-limiting examples of which include Irritable Bowel Disease (IBD), Irritable Bowel Syndrome (IBS), Crohn's Disease, colitis, biliary colic, renal colic, inflammatory disorders of the GI tract, cancers of the GI tract (including colorectal cancer and adenocarcinoma of the small bowel) and diabetes.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

The following materials and methods were used in the studies described in the Examples:

Materials and Methods

GIT-ORIS Device Manufacturing

GIT-ORIS interface device was manufactured by laser cutting holes (VLS6.60 from Universal Laser Systems) identical to standard 6, 12, 24, 48, 96, 384, 1536 well plate designs using acrylic sheets with 1 cm thickness (McMaster-Carr). A recess on the longer sides was milled by the laser to separate plates by hand and allow a robotic arm to hold the plates. Black, white or translucent acrylic was used depending on the final assay read out. Nickel plated, axially magnetized N52 grade magnets (2.28 lb force/magnet) (K&J Magnetics, Inc), were embedded in both plates and enabled tissue compression in between to ensure tight assembly for robotic handling and no well-to-well leakage. The magnets needed to be positioned at the outer edge as well as in the middle of the plate with 9 magnets per plate exerting a total force of 20.52 lbs. The holes on the bottom surface of the plates were sealed with optically clear Microseal ‘C’ Film (Biorad MSC1001).

Tissue Dissection and GIT-ORIS Preparation

All animal tissue procedures were conducted in accordance with protocols approved by the Massachusetts Institute of Technology Committee on Animal Care. Small intestinal tissue was isolated from freshly procured intact gastrointestinal tracts from pigs from selected local slaughterhouses. The intact gastrointestinal tract was harvested after euthanization and bleeding of the animal and put on ice immediately afterwards. Tissue dissection was performed 1 hour after isolation. For intestinal perfusion and absorption experiments, jejunual tissue was used. Jejunal tissue was defined as 50 cm away from the pylorus. The difference between the jejunum and ileum was determined based on anatomical location, the structural differences of the tissue, differences in blood supply, fat deposition, and presence of lymphoid tissue. A stretch of the tissue was cut out of the GI tract and dissected longitudinally. The tissue was washed in a series of saline solutions supplemented with 5% Antibiotic-Antimycotic solution (Cat. nb. 15240062, Thermo Fisher Scientific) under sterile conditions. The tissue was then either mounted on the GIT-ORIS device. For intestinal perfusion and absorption experiments, the bottom of the 2-plate system was prefilled with transport buffer supplemented with 5% Antibiotic-Antimycotic solution. Then dissected intestinal tissue was carefully placed on top without creating any air bubbles that would obstruct the transport. Then the upper plate was placed on top. The magnetic force immediately aligns the plates and maintains the position of the set up without any further requirements. Screening experiments were then either conducted immediately or the next day. During overnight incubation the tissue was stored at 4° C. and warmed up to 37° C. 2 hours prior to the experiment. For expression analysis that require ex vivo cultivation of the tissue, GIT-ORIS receiver well was prefilled with serum-free cell culture media (Advanced DMEM/F-12 (Lifetechnologies, cat. no. 12634028) in order to generate a liquid-air interface cultivation. The tissue was then incubated at 37° C. for ex vivo cultivation without supplemental gas.

Automated GIT-ORIS Perfusion and Absorption Screening Experiments

Intestinal perfusion experiments using the system were conducted within 24 hours of ex vivo cultivation unless otherwise noted. Formulation samples were prepared using a liquid handling station (Evo 150 liquid handling deck, Tecan) that followed a protocol to mix the pre-prepared excipient master plate, containing the diverse compound library (see Excipient preparation section), 10 times. After pre-mixing, a volume of 150 μL per well was transferred into an intermediate 96-well plate prefilled with 30 μL per well of a freshly prepared concentrated AON working solution in PBS to achieve a final total concentration of 25 μM AON and 83 mg/mL compound. In order to achieve successful mixing and generate reproducible dispersions the samples were mixed 60 times using liquid handling station. Then GIT-ORIS 96-well plate device was moved from the microwell plate hotel (Peak Analysis & Automation) to the liquid handling station automatically using a 6-axis industrial robot (Staubli) and 50 μL per well was transferred from the intermediate well plate into the GIT-ORIS 96-well plate device. Immediately afterwards, the robotic arm transferred the GIT-ORIS well plate to a microplate reader (Infinite® M1000 PRO, Tecan) for simultaneous FAM fluorescence signal detection in the receiver and donor chamber (initial time point). Then, the signal was detected kinetically over a 4 hour incubation period in 20 minutes intervals by automatic transfer by the robotic arm between the microwell plate hotel and the microplate reader. Afterwards, for intestinal absorption measurements, the liquid was removed from the receiver and donor well of the GIT-ORIS device and the tissue was washed with a heparin (medium molecular weight, Sigma) solution (0.1 mg/ml in PBS) followed by 3 washes with PBS. Then the plate was again inserted in the microplate reader and the fluorescence intensity of the apical and basal side of the tissue was measured. All experiments, including sample incubation, were performed at room temperature.

Locked Nucleic Acids (AON)-Containing Gapmers Synthesis

Locked nucleic acid oligonucleotides were synthesized on solid support by the phosphoramidite method using a synthesis cycle consisting of detritylation, coupling, sulphurization and capping, which was repeated until the full length product was obtained. After completion of solid phase synthesis, the oligonucleotide was cleaved from the support and deprotected by suspending the solid support in concentrated aqueous ammonia at 55 degrees Celsius for 4 hours. Fluorescein (FAM) labels were incorporated as a phosphoramidite during solid phase synthesis, using 6-[(3′,6′-Dipivaloylfluoresceinyl)-carboxamido]-hexyl-1-O-[(2-cyanoethyl)-(N,N-diisopropyl)]-phosphoramidite purchased from link technologies in the final coupling cycle. AlexaFluor647 labels were synthesized by conjugation of AlexaFluor647 NHS ester purchased from Life Technologies Europe to aminohexyl labelled oligonucleotides. The aminohexyl label was incorporated during solid phase synthesis as a phosphoramidite using 6-(Trifluoroacetylamino)hexyl-[(2-cyanoethyl)-(N,N-diisopropyl)]-phosphoramidite purchased from link technologies in the final coupling cycle. After cleavage and deprotection of the aminohexyl oligonucleotide, the ammonia was removed in vacuo, and the oligonucleotide was dissolved in 1 mL water, and filtered through a 0.45 μm syringe filter. Hereafter the aminohexyl labelled oligonucleotides were precipitated as lithium salt by addition of 5 mL 2% (w/v) LiClO₄ in acetone to prepare for conjugation. The precipitate was recovered by centrifugation, and the supernatant was decanted. The resulting oligonucleotide pellet was dissolved in 200 μL 100 mM sodium carbonate buffer pH 8.5. The concentration was determined by OD(260). 0.2 μmol of the oligonucleotide from this solution was added to 1 mg alexaFluor647 NHS ester dissolved in 50 μL anhydrous N,N-Dimethylformamide. The conjugation was allowed to proceed in the absence of light overnight. Hereafter the product was precipitated from the solution by addition of 1 mL 2% (w/v) LiClO₄ in acetone. The precipitate was recovered by centrifugation, and redissolved in 1 mL MilliQ water filtered through a 0.45 μm syringe filter. FAM and AlexaFluor647 labelled oligonucleotides were purified by preparative RP-HPLC on a Jupiter C18 column with a 5-60% acetonitrile gradient in 0.1M ammonium acetate pH 8 in milliQ water over 15 min with a flowrate of 5 mL/min. Fractions were collected based on absorption of the fluorophore (647 nm for AlexaFluor647 labels, and 495 nm for FAM labels). The fractions containing desired product were concentrated in vacuo and dissolved in PBS buffer. Unlabeled oligonucleotides were purified by tangential flow filtration. The resulting aqueous solution of oligonucleotide was lyophilized resulting in the oligonucleotide as a white powder. All products were analyzed by UPLC-MS to confirm identity and purity.

Reagents

δ-decalactone, (−)-terpinen-4-ol, (±)-4-methyloctanoic acid, 1,1-aminoundecanoic acid, 1-adamantylamine, 2,2-bis (hydroxymethyl) propionic acid, 2,2-dimethylbutyric acid, 2-butyloctanoic acid, 2-ethylbutyric acid, 2-ethylhexanoic acid, 2-hydroxy 2-methylpropiophenone, 2-methylhexanoic acid, 2-phospho-L-ascrobic Acid trisodium salt, 2-propylpentanoic acid/Valproic acid, 3-(trimethylsilyl)-1-propanesulfonic acid, 3,3-dimethylbutyric acid, 3,4-dihydroxy 1-phenyl alanine, 3,4-dihydroxy 1-phenyl alanine, 3,7-dimethyl-6-octenoic acid, 4 Arm PEG, 4-(dimethylamino)pyridine, 4-ethyloctanoic acid, 4-methylnonanoic acid, 4-methylvaleric acid, 6-O-palmitoyl-L-ascorbic acid, 8 Arm PEG, acesulfame k, acetyl salicylic acid, adipic acid, advan hydrothane, agarose, albumin (bovine serum), alginic acid ammonium salt, alginic acid calcium salt (brown algae), alginic acid potassium salt, alginic acid sodium salt, alginic acid sodium salt (brown algae), alpha cyclodextrin, alpha-D-glucose, aluminum hydroxide, aluminum lactate, aluminum oxide, aluminum silicate, aluminum silicate, aluminum sulfate hydrate, ammonium aluminum sulfate dodecahydrate, ammonium carbonate, ammonium chloride, ammonium iron (III) citrate, ammonium molybdate, beta-alanine, aarium sulfate, bentonite, benzoic acid, benzophenone, beta-cyclodextrin, beta-glycero phosphate disodium salt, caffeine, calcium acetate hydrate, calcium carbonate, calcium chloride, calcium citrate (tetrahydrate), calcium D-gluconate, calcium fluoride, calcium iodate, calcium L-lactate hydrate, calcium phosphate amorphous nanopowder, calcium phosphate dibasic, calcium phosphate monobasic, calcium silicate, castor oil, chitosan (high mw), chloroquine diphosphate salt, choline bitartarate, choline chloride, citric acid, corn oil, cottonseed oil, cysteamine, D(−)fructose, D(+)cellobiose, D(+)glucose, D(+)mannose, D(+)trehalose (dihydrate), dextran 70 kDa, dextrose, diethylene glycol, DL-lactic acid, DL-tartaric acid, D-mannitol, dodecanedoic acid, D-sorbitol, D-tryptophan, Dynasan 118 (microfine), edetate disodium, edta, egta, ethyl formate, ethylene diamine tetraacetic acid, ethylparaben, EUDGRAGIT® E PO, EUDGRAGIT® NM 30D, EUDGRAGIT® RL PO, EUDGRAGIT® S100, EUDRAGIT® L 100-55, EUDRAGIT® RS PO, gelatin, gelatin from cold water fish skin, geraniol, glycerin, glycerol phosphate calcium salt, glycine, glycocholic acid, guar, HEPES, heptanoic acid, hydroxyapatite, hydroxymethyl polystyrene, Indomethacin, iron (II) chloride (tetrahydrate), iron (II) D-gluconate (dihydrate), iron (III) oxide, kaolin, Koliphor® EL, Kollidon® 25, Kollidon® VA 64, Kollidon® 12PF, Kollidon® P188, Kollidon® SR, Kollidon® P407, Kollidon® RH40, Kolliphor® EL, L-lysine, L(+) arabinose, L-arginine, L-ascorbic acid, L-cysteine hydrochloride, lecithin, L-glutamic acid, L-histidine, lithium bromide, lithium hydroxide, L-phenylaline, L-proline, magnesium carbonate, magnesium D-gluconate hydrate, magnesium hydroxide, magnesium oxide, magnesium phosphate dibasic trihydrate, magnesium sulfate, manganese sulfate monohydrate, meglomine, methyl paraben, mineral oil, mucin (porcine stomach), neohesperidin, N-hydroxysuccinimide, nonanoic acid, octanoic acid, parafin wax, PDMS-bis(3-aminopropyl) terminated, PDMS-co-methyl (3-hydroxypropyl) siloxane] graft-mPEG, PDMS-graft polyacrylates, peanut oil, PEG 20 kDa, PEG 3350 da, PEG 35 kDa, PEG 400 Da, PEG 400 kDa, PEG diacrylate, PEG methylether, PEG-block-PEG-Block-PEG, pepsin (porcine gastric mucosa), pimelic acid, Pluronic F-127, Pluronic F-68, Pluronic P85, poly (sodium 4-styrene sulfonate), poly(ethylene-co-glycidyl methacrylate), poly(ethylene-co-vinyl-acetate), poly(methyl methacrylate-co-methacrylic acid) 34 kda, poly(propylene glycol) diglycidyl ether, polyacrylic acid, polyethylene-block-PEG, polyethylenimine 800 da, potassium acetate, potassium bromide, potassium carbonate, potassium chloride, potassium citrate (tribasic), potassium disulfite, potassium gluconate, potassium iodate, potassium nitrate, potassium phosphate, potassium phosphate (dibasic), potassium phosphate (monobasic), potassium pyrophosphate, potassium silicate, propyl gallate, pyridoxine, pyridoxine hydrochloride, R-(+)limonene, saccharin, sesame oil, Sigma 7-9 (tris base), silica gel, sodium acetate (trihydrate), sodium azide, sodium bicarbonate, sodium cacodylate (trihydrate), sodium carbonate, sodium chloride, sodium citrate (dihydrate), sodium dodecyl sulfate, sodium fluoride, sodium gluconate, sodium glycholate, sodium glycochenodeoxycholate, sodium hyaluronate, sodium hydroxide, sodium iodide, sodium malonate (dibasic), sodium metabisulfite, sodium nitrite, sodium perchlorate hydrate, sodium perchlorate monohydrate, sodium phosphate (dibasic), sodium phosphate monobasic, sodium pyrophophate tetrabasic, sodium salicylate, sodium sulfite, sodium tartrate dihydrate (dibasic), sodium taurocholate hydrate, sodium tetraborate decahydrate, sodium-L-ascorbate, Soluplus, soybean oil, Span 80, starch (from corn), starch (soluble), stearic acid, suberic acid, succinic acid, sucrose, sucrose octa-acetate, Synperonic F108, talc, tannic acid, tauchloric acid, taurochenodeoxycholate, taurodeoxycholate, terephthalic acid, terephthalic acid, tetrabutyl ammonium phosphate (monobasic), thimerosal, thimesosol, tin (II) chloride, tragacanth, tri sodium citrate dehydrate, triacetin, trimesic acid, tris (hydroxymethyl) amino-methane, TritonX100, turmeric, Tween® 80, Tween® 20, Tween® 28, tyramine, vanillin, vegetable oil, xylitol, γ-decalactone, zinc acetate, zinc carbonate (basic), zinc chloride, zinc citrate dehydrate, zinc oxide, zinc sulfate monohydrate, ε-caprolactam, ε-caprolactone, w-pentadecalactone. Oils: Bay oil, canola oil, soybean oil, lovage oil, dillweed oil, cardamom oil, lemongrass oil, tea tree oil, jojoba oil from Simmondsia chinensis, cinnamon oil (ceylon type, nature identical), Eucalyptus oil, garlic oil (chinese), coriander oil, cognac oil, celery seed oil, corn oil, cedar oil, lard oil, bergamot oil, palm oil, castor oil, guaiac wood oil, ginger oil, geranium oil (chinese), nutmeg oil, peppermint oil, epoxidized soya bean oil, wheat germ oil, palm fruit oil, jojoba oil, tung oil, sandalwood oil, fennel oil, olive oil, linseed oil, menhaden fish oil, croton oil, peanut oil, anise oil, coffee oil, fusel oil, patchouli oil, lemon oil, spearmint oil, vegetable oil, sesame oil, flax seed oil, rosemary oil, mandarin oil, Cassia oil, cade oil, citronella oil (java), coconut oil, safflower oil, sunflower seed oil, clove oil, rapeseed oil from Brassica rapa, cedar leaf oil, avocado oil, thyme oil, lavender oil, orange oil, mineral oil, sunflower oil, wintergreen oil, lime oil, pine needle oil, birch oil, cypress oil, clove bud oil, cottonseed oil.

Preparation of LNA Formulations

All formulations were prepared as mixtures containing 83 mg/mL excipient and 20 microM LNA in PBS buffer (Dulbecco's phosphate buffered saline without calcium chloride or magnesium chloride). The mixtures were mixed via automated pipetting and then added directly onto the tissue surface. No other sample treatment was performed. Oil emulsions were prepared using 83% (volume percent) oil and 17% (volume percent) aqueous PBS buffer solution (Dulbecco's phosphate buffered saline without calcium chloride or magnesium chloride) containing 20 microM LNA. For the emulsion process, LNA was added in buffer solution, then oil was added and the entire solution was mixed 60 times by pipetting via a liquid handling station. This generated an oil-water emulsion that was then immediately used as the formulation.

In Vivo Analysis in Porcine Model

All animal experiments were conducted in accordance with protocols approved by the Massachusetts Institute of Technology Committee on Animal Care. Sample size was guided by prior proof-of-concept studies in the area of gastrointestinal drug delivery and electronics. For in vivo drug delivery studies female Yorkshire pigs between 50 and 80 kg in weight were used. Before every experiment, the animals were fasted overnight. On the day of the procedure the morning feed was held. The animals were sedated with an intramuscular injection of telazol (tileramine/zolazepam) 5 mg/kg, xylazine 2 mg/kg and atropine 0.04 mg/kg. After complete sedation, the small intestine was accessed surgically. A cylindrical shaped device with an L-shaped rim (Lid of static vertical glass diffusion cell used with 1.77 cm² surface area from PermeGear) coated with a layer of Carbopol (Carbopol 971PNF, Lubrizol) on one side was then inserted in the luminal side of the jejunum via a small longitudinal incision in the jejunum. The incision was performed distal to the blood vessels without creating major bleeding. By pressing the device on the tissue for 60 seconds we obtained a seal between the device and the luminal side of the jejunum because of the mucoadhesive properties of Carbopol. 2 mL of sample volume was then added in each device on the tissue inside the cylindrical device. After 2 hours incubation the device was removed and biopsy samples were obtained which were then immediately fixed in 4% (v/w) formalin in PBS for 2 days and then processed histologically as described in the previous section.

Expression Analysis

Knock-down efficiency for each formulated and/or unformulated AON was determined through analyzing the expression level of corresponding targeted gene using real-time quantitative PCR. Briefly, total RNA from each tissue sample was extracted and purified with Quick-RNA Plus™ (Zymo Research) followed with reverse transcription into cDNA by High-Capacity cDNA reverse transcription kit (ThermoFisher Scientific). Target genes were amplified by FAM-labeled primer (Bio-rad), and phosphoglycerate kinase 1 (PGK1) was chosen as the internal control, which was amplified by VIC-labeled primer (Bio-rad). The PCR reaction was measured with LightCycler® (Roche). The relative quantification of gene expression was performed according to the ΔΔ-C_{T} method. The gene expression level of non-treated tissue was used as baseline. We also PCR amplified two long fragments (312 and 836 bp) from genomic DNA, which was isolated and purified with Quick-DNA Plus™ (Zymo Research), in order to prove the high quality of tissue samples after AON treatment and culturing for 24 and 48 hours.

In Situ Hybridization Immunohistochemical Staining

Tissue explants were fixed in 4% (v/w) formalin in PBS for 2 days at 4° C. Then dehydration and paraffin embedding was performed followed by tissue sectioning. For the resulting paraffin embedded tissue slides, dewaxing was conducted according to standard protocols followed by staining procedure. Tissue slides were incubated in proteinase K buffer for five minutes in a 37° C. incubator and then washed for two minutes with PBS. For buffer preparation (pH 8) the following reagents were used: 5 ug/ml proteinase k (Sigma P4850), 50 mM Trizma Hydrochloride solution and 5 mM ethylenediaminetetraacetic acid. The slides were then fixed with 10% (v/w) formalin in PBS for five minutes and washed three times with PBS for five minutes each. After, the slides were incubated through a graded acetic anhydride in 1M triethanolamine series for each concentration (0.25%, 0.50% (v/v)) for five minutes each on a stirring plate. This was followed by three washes with PBS for five minutes each and incubation in pre-warmed hybridization buffer for thirty minutes in the hybridization oven at 67° C. Hybridization buffer consisted of 1×Denhardts Solution (Sigma D2532), 500 ug/mL yeast tRNA (Sigma 10109495001), 50% formamide and 5×SSC (Sigma S6639). For probe preparation, FAM-labeled AON 12798 was heated to 90° C. for four minutes, immediately placed on ice to prevent annealing and diluted in hybridization buffer before being added to the remainder of the buffer for final probe concentration of 30 nM. Slides incubated in the buffer for thirty minutes and then washed three times with pre-warmed 0.1×SSC for five minutes. Both hybridization and washing steps occurred in the hybridization oven at 67° C. After, the slides were immersed in 3% (v/w) hydrogen peroxide in PBS for 10 minutes, washed three times with PBS for five minutes each and blocked with TNB solution (pH 7.5) for fifteen minutes. For TNB preparation the following reagents were used: 0.1 M Trizma Hydrochloride solution, 0.15 M sodium chloride and 0.5% blocking reagent (Perkin Elmer FP1020).

Microscopy Analysis

Light microscopy analysis of histology slides was conducted using an EVOS FL Cell Imaging System with 10× or 20× air objectives. Fluorescent samples were analyzed using an Ultra-Fast Spectral Scanning Confocal Microscope (Nikon A1R) with a Galvano scanner and 20× air or 60× oil immersion objectives. Resulting raw images were analyzed with NIS-Elements C software and ImageJ. If needed the brightness and contrast of images was adjusted. This was done consistent for the entire set of images in the same experiment. No further image processing was applied.

Mucus Diffusion Analysis

Intestinal mucus was freshly harvested from the jejunum of pigs by gently squeezing an intestinal segment longitudinal by hand. Then the harvested content was transferred into a 384 well plate (Greiner Sensoplate™ glass bottom multiwell plates) (50 μL/well). The plate was then used for mucus diffusion experiments immediately by placing a solution of fluorescently labelled AON formulation (40 μL/well) on top of the mucus layer. For validation experiments the AON formulation was homogenized with the mucus to generate a homogeneous solution. 3D stacks of each well was then obtained by using an Ultra-Fast Spectral Scanning Confocal Microscope (Nikon A1R) with a resonant scanner and a 4× air objective. The image stack height was set to cover the entire mucus layer. In order to compensate signal loss of signal in the mucus layer, a z-correction function was programmed that adjusted the laser power as a function of sample depth in order to ensure constant fluorescence intensity throughout the mucus depth. 3D stacks were then obtained over time. The displacement of fluorescence signal over time in mucus in 3D was then used in order to estimate the mucus diffusion by analysis in MATLAB.

Cell Culture and AON Formulation Uptake Analysis

HT29-MTX-E12 cells were purchased from European Collection of Authenticated Cell Cultures (ECACC) (Cat. Nb. 12040401) and cultured under standard cultivation conditions (37° C., 5% CO₂) in DMEM high glucose pyruvate (Lifetechnologies, cat. no. 11995-065) with 1% Gibco MEM Non-Essential Amino Acid Solution (Lifetechnologies, Cat #11140-050), 1% Pen/Strep (Lifetechnologies, Cat #15140122), 10% FBS (heat inactivated) (Lifetechnologies, Cat #10082-147). C2BBe1 [clone of Caco-2] cells were purchased from ATCC (ATCC® CRL-2102™) and cultured under standard cultivation conditions (37° C., 5% CO₂) in DMEM high glucose pyruvate (Lifetechnologies, cat. no. 11995-065) with 1% Human Transferrin-insulin-Selenium (ITS-G) 100× (Lifetechnologies, Cat #41400-045), 1% Pen/Strep (Lifetechnologies, Cat #15140122), 10% FBS (heat inactivated) (Lifetechnologies, Cat #10082-147). All cells tested negative for mycoplasma contamination. For uptake screening experiments, cells were seeded in 96 well plates (Corning® 96-well plates, clear bottom, Corning) at 30,000 cells per well. Next day, a PBS solution of FAM-(AON)-containing gapmer against the target PTEN (2.5 μM final concentration) formulated with various excipients (10 mg/ml final concentration) was added in a 1:1 ratio (100 μL AON formulation solution to 100 μL existing media) and incubated for 1 hour followed by a washing steps and subsequent spectrophotometric detection of FAM signal using a multiplate reader (Tecan M1000).

Statistical Analysis

Correlation matrix for formulation screening analysis was calculated using two-tailed Pearson correlation function. Statistical analysis of target gene expression results was conducted by a one-way ANOVA followed by a Bonferroni and Tukey test.

### Example 1: Locked Nucleic Acids (AON)-Containing Gapmers

In studies described in the Examples, locked nucleic acids (AON)-containing gapmers have been used in which the ribose ring is “locked” by a methylene bridge connecting the 2′-O atom and the 4′-C atom. This modification promotes a rigid RNA-like structure which enables nuclease resistance and dramatic increases in binding affinity to the target. Locked nucleic acids (LNA) have been described in the art, see e.g., Hagedorn et al. (2017) Drug Discov. Today 23:101-114.

LNA sequences that were used in the studies are shown below in Table 1 and in SEQ ID NOs: 1-8, respectively:

TABLE 1
LNA Sequences

| LNA Name | LNA Sequence |
|---|---|
| LNA against HIF-1 alpha | 5′-GCaagcatcctGT |
| FAM-LNA against HIF-1 alpha | 5′-[FAM]S1GCaagcatcctGT |
| LNA against PTEN (Version #1) | 5′TCActtagccattGGT |
| LNA against PTEN (Version #2) | 5′ACttagccatTG |
| FAM-LNA against PTEN (Version #2) | 5′-[FAM]ACttagccatTG |
| FAM-LNA against PTEN (Version #1) | 5′-[FAM]TCActtagccattGGT |
| Alexa647-LNA against PTEN (Version #1) | 5′[Alexa647]TCActtagccattGGT |
| Alexa647-LNA against HIF-1 alpha | 5′-[Alexa647]S1GCaagcatcctGT |

Uppercase letters denote LNA nucleotides and lowercase letters denote DNA nucleotides. For LNA nucleotides, all cytosines were 5-methyl cytosines. All intemucleoside linkages were phosphorthioates. S1 denotes hexaethyleneglycol linker, [Alexa647] denotes Alexa647 NHS ester conjugated to aminohexyl linker and [FAM] denotes fluorescein.

The effects of LNA gapmers on the expression of the target genes (HIF-1 alpha and PTEN) was assessed by rtPCR expression analysis using the PTEN and HIF-1 alpha primers shown below in Table 2 and in SEQ ID NOs: 9 and 10, respectively. The housekeeping gene PGK-1 was used as a control, the primer for which is also shown below in Table 2 and in SEQ ID NO: 11.

TABLE 2
Primer Sequences

| Target | Probe | |
|---|---|---|
| Name | Description | Probe Sequence |
| PTEN | Tagman-Fam- | TCCAATGTTCAGTGGCGGAACTTGCAATCCTCA |
| | MGB probe: | GTTTGTGGTCTGCCAGCTAAAGGTGAAGATATA |
| | Ss03820741 | TTCCTCCAATTCAGGACCCACACGACGGGAAGA |
| | (ThermoFisher), | CAAGTTCATGTACTTTGAGTTCCCTCAGCCCATT |
| | length: 97 bp. | GCCTGTGTGTGGTGACATCAAAGTAGAGTTCTT |
| | exon location: 7 | CCACAAACAGAACAAGATGCTAAAAAAGGACA |
| | | AAAT (SEQ ID NO: 10) |
| HIF-1 | Tagman-Fam- | TATGAGCTTGCTCATCAGTTGCCACTTCCCCAT |
| alpha | MGB probe: | AATGTGAGCTCACATCTTGATAAGGCTTCTGTT |
| | Ss03390447 | ATGAGGCTTACCATCAGCTATTTGCGTGTGAGG |
| | (ThermoFisher), | AAACTTCTAGATGCTGGTGATTTGGATATTGAA |
| | length: 111 bp, | GATGAAATGAAGGCACAGATGAATTGTTTTTAT |
| | exon location: | TTGAAAGCCTTGGATGGTTTTGTTATGGTACTC |
| | 2-3 | ACAGATGATGGTGACATGATTTATA (SEQ ID |
| | | NO: 11) |
| PGK-1 | Tagman-VIC- | GTCATCCTGTTGGAGAACCTTCGCTTTCATGTG |
| | MGB probe: | GAGGAAGAAGGGAAGGGAAAAGATGCTTCTGG |
| | Ss03389144 | GAGCAAGGTTAAAGCTGATCCAGCCAAAATAG |
| | (ThermoFisher), | AAGCCTTCCGAGCTTCACTTTCCAAGCTAGGGG |
| | length: 66 bp, | ATG (SEQ ID NO: 11) |
| | exon location: | |
| | 2-3 | |

### Example 2: In Vitro System for High Throughput Screening of Formulations for Gastrointestinal Delivery

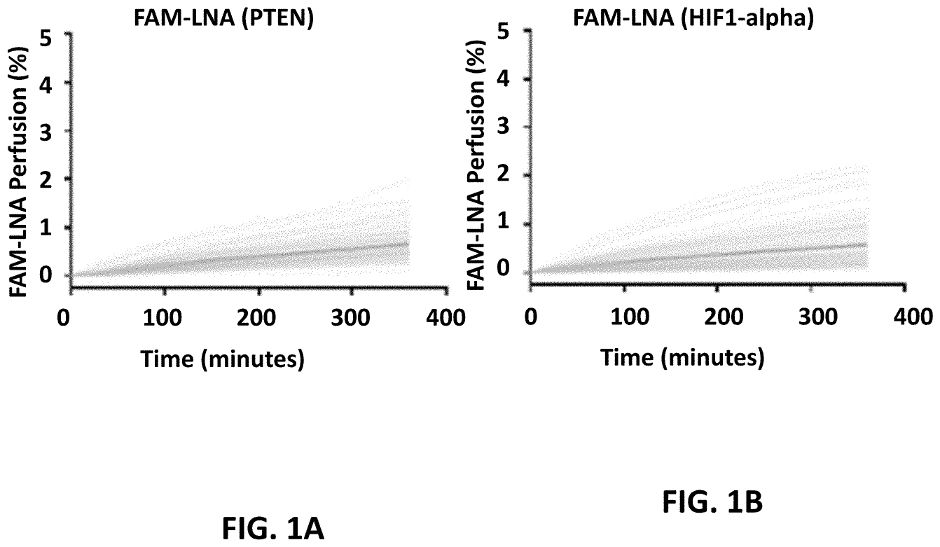

This example describes a system that enables high throughput screening of fully intact ex vivo cultured GI tissue derived from pigs, called the gastrointestinal tract organ robotic interface system (GIT-ORIS). This system is described in detail in U.S. Patent Publication No. US 2019/0064153, filed Mar. 23, 2018 (herein incorporated in its entirety by this reference) and also described above in the Materials and Methods section. The GIT-ORIS relies on custom designed plates that confine GI tissue in sealed wells by magnetic compression. This system was specifically designed to fully interface with a robotic screening platform including real-time detection by a plate reader without disassembly of the device. Methods have been developed that enable simultaneous automated high throughput detection of fluorescently conjugated AONs that accumulated or perfused through the GI tissue. Automated high throughput kinetic perfusion analysis with the GIT-ORIS was found to be highly reproducible as assessed by measurements of 6-Carboxyfluorescein (FAM) labelled oligonucleotides over different animal batches and parts of the jejunum. FIGS. 1A-1B show the results of the kinetic perfusion analysis of FAM-labelled AON-containing gapmers against either HIF-1 alpha or PTEN over 6 hours with 500 samples each (n=170). The results demonstrate effective perfusion of both AONs.

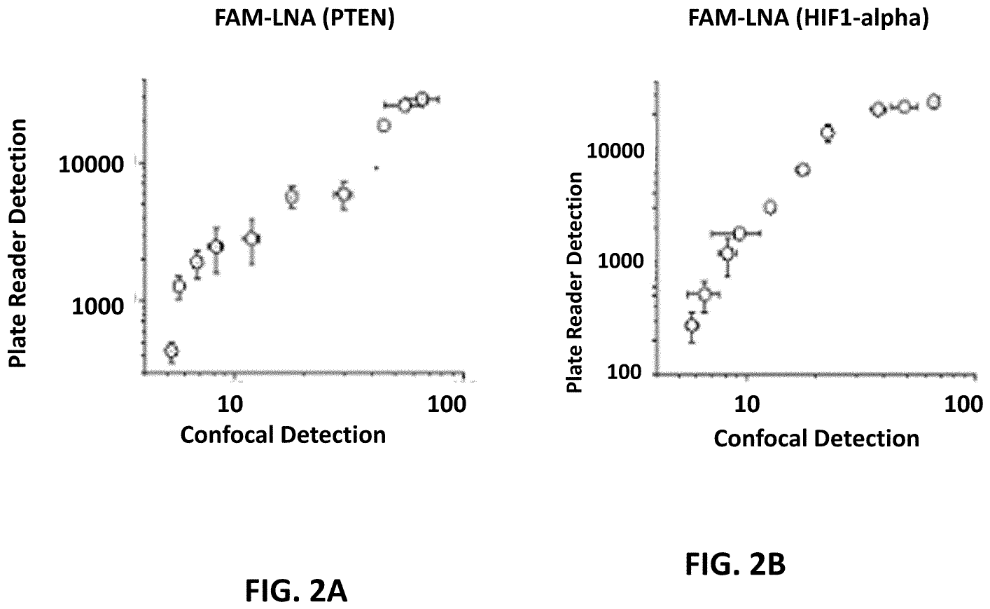

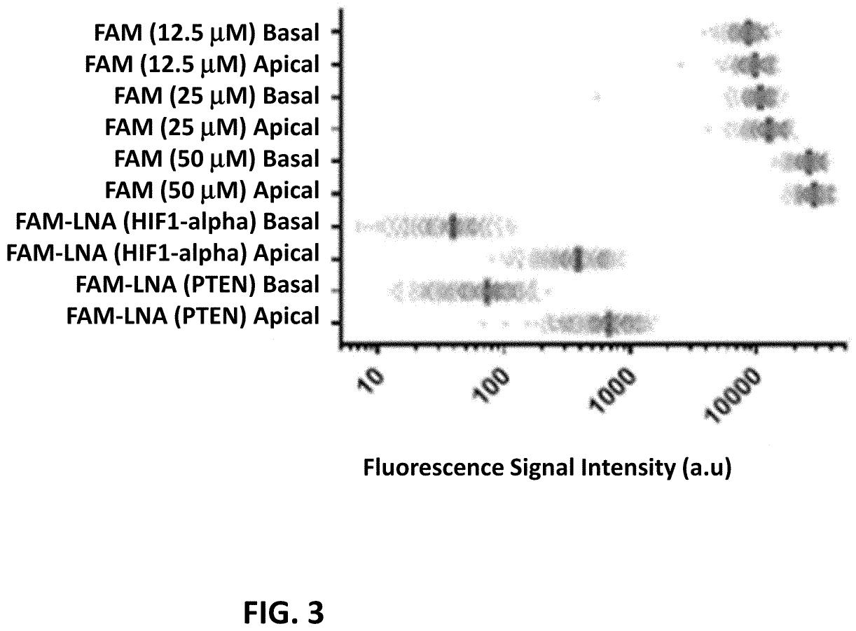

A high-throughput compatible spectrophotometric-based read-out method to measure FAM-AON tissue was developed and validated by confocal microscopy-based signal detection. Comparison of confocal based detection and spectrophotometric detection of intestinal tissue accumulation of locked nucleic acids (AON)-containing gapmers showed a linear correlation, as demonstrated in FIGS. 2A-2B. Automated high throughput apical and basal tissue accumulation measurements of FAM label only and FAM-AON across multiple animal batches and various segments of the jejunum demonstrates low variability and high reproducibility, as shown in FIG. 3.

### Example 3: Screening of Formulations Using In Vitro GIT-ORIS System

In this example, the in vitro system described in Example 2 was used to screen formulations of the AONs described in Example 1 for intestinal perfusion and absorption.

Screening experiments were conducted using formulations of FAM labelled AONs against hypoxia-inducible factor 1 alpha (HIF-1 alpha) and phosphatase and tensin homolog (PTEN) respectively and measuring intestinal perfusion and tissue absorption in real time simultaneously. The HIF-1 alpha and PTEN AONs were initially formulated using a custom designed diverse chemical compound library (285 compounds) that represents a wide range of chemical properties to identify compounds that modulate local intestinal tissue uptake for topical treatment (defined as “intestinal absorption”) or permeation through the intestinal tissue for potential enhanced systemic bioavailability (defined as “intestinal perfusion”) of the AONs.

The results for screening of the chemical compound library are summarized in the heatmap analysis shown in FIG. 4. The screening data revealed a range of compounds that showed a several-fold increase in either intestinal perfusion or absorption enhancement or both.

The results of the chemical compound screen indicated that oil emulsion based AON formulations were promising enhancers of both intestinal tissue perfusion and absorption. Therefore, another screen was conducted based on 213 oil-emulsion formulations for two FAM conjugated AONs against either HIF-1 alpha and or PTEN. For this formulation screening experiment, a library of 71 different organic oils as assembled that was then combined with 3 different emulsifiers (Soluplus®, Pluronic F127 and Tween® 20) through a standardized dispersion process. The results for screening of the oil emulsion library are summarized in the heatmap analysis shown in FIG. 5. Indeed, the screening results reveal a high number of newly discovered formulations that act as enhancers of intestinal absorption and perfusion.

Interestingly, AON absorption and perfusion enhancements are dependent on the specific oil composition as well as the emulsifier used. The data from the diverse chemical compound screen reveals little correlation between intestinal tissue perfusion and absorption AON enhancement. In addition, the permeability versus absorption correlation appears to be highly dependent on the AON sequence (Pearson Coefficient r=0.05 permeability vs. apical absorption, r=0.16 permeability vs. basal absorption for AON against PTEN; r=0.44 permeability vs. apical absorption, 0.63 permeability vs. basal absorption for AON against HIF-1 alpha). In contrast, the more homogeneous oil-emulsion formulation library shows a clear correlation between perfusion and absorption AON enhancement for both AONs tested (Pearson Coefficient r=0.64 permeability vs. apical absorption, r=0.69 permeability vs. basal absorption for AON against PTEN; r=0.73 permeability vs. apical absorption, 0.75 permeability vs. basal absorption for AON against HIF-1 alpha). Furthermore, AON formulations using the diverse chemical compound library show differences in intestinal tissue absorption and perfusion depending on the AON sequence. Interestingly, AON oil emulsifier formulations for PTEN and HIF-1 alpha show higher correlation in intestinal tissue absorption and perfusion between the different AON sequences used.

Overall, these observations demonstrate that the effect of formulations on the AON intestinal absorption or perfusion is specific to the AON sequence and that this effect is more pronounced in certain formulations than others. This observation is expected to be highly relevant for the oral formulation of other oligonucleotide drugs beyond LNA-containing gapmer AONs as well as other active pharmaceutical ingredient classes. Furthermore, a poor correlation was observed in formulation dependent uptake of AON between cell line monolayers compared to ex vivo intestinal tissue. This may be due to differences at the level of drug transporter expression (Hayeshi et al. (2008) Eur. J. Pharm. Sci. 35:383-396) as well under-representation of the complex intestinal architecture and milieu (Artursson et al. (1993) Pharm. Res. 10:1123-1129; Collett et al. (1997) Pharm. Res. 14:767-773).

### Example 4: Mucus Diffusion Analysis Using 4D Confocal Imaging

To investigate the effect of formulations on the diffusion of AON through the intestinal mucus barrier, a 4D confocal imaging technique was developed that enables evaluation of the lateral and spatial displacement of fluorescently labelled AON in native intestinal mucus over time. While the underlining concept is similar to previously reported techniques (Lai et al. (2009) Adv. Drug Deliv. Rev. 61:158-171), this assay has the advantage of being able to measure multiple samples simultaneously and can be used in a 96 or 384 well plate format.

The detection of FAM-AON homogeneously distributed in freshly harvested native porcine intestinal mucus was established. Addition of FAM-AON solution on top of the mucus layer followed by 4D confocal imaging showed clear signal displacement over time and no effect of photobleaching. A dose-dependent increase in fluorescence intensity demonstrated proportional signal increase with increasing FAM-AON concentration within the 3D mucus layer over time.

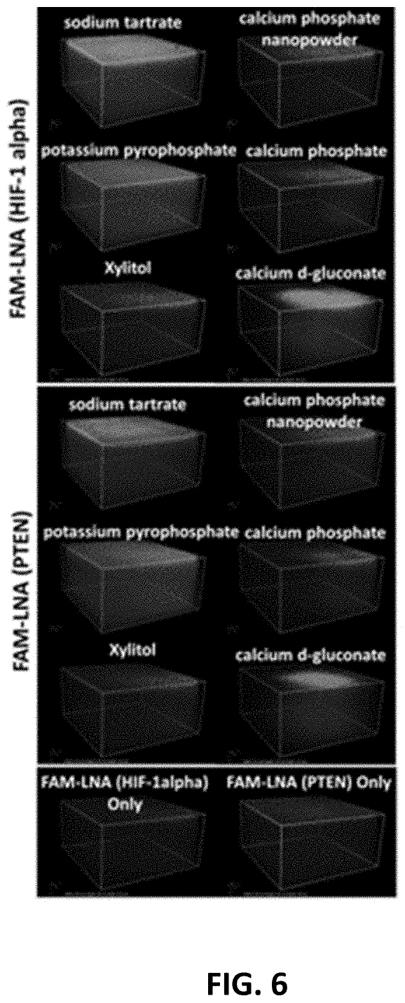

The diffusion of various formulations of FAM-AONs targeting either HIF-1 alpha or PTEN was then measured through the mucus. Representative images of FAM fluorescence intensity of FAM-LAN (HIF-1 alpha) and FAM-LAN (PTEN) formulations placed on top of mucus layer and incubated for 75 minutes are shown in FIG. 6. Fluorescence signal displacement was used to assess diffusion of FAM-AON into the mucus layer.

The diffusion analysis using 4D confocal imaging allowed for identification of several formulations that showed a multiple fold increase in mucus diffusion as compared to the FAM-AON only control. This subpanel is summarized in FIG. 7, in which the results are compared to the change in intestinal permeability and absorption using the GIT-ORIS system with intestinal mucus layer intact versus washed away. The results in FIG. 7 are summarized as fold changes compared to the non-formulated control in a color-coded heatmap.

### Example 5: Further In Vitro Analysis of Selected Formulations

Based on the screening results described in Examples 3 and 4, a subpanel of AON formulations was selected for further in-depth analysis. As part of this, AONs targeting PTEN and HIF-1 alpha were conjugated to Alexa 647 (recognized for its superior sensitivity and specificity, as described in Buschman et al. (2003) Bioconjugate Chem. 14:195-204). Indeed, dose-dependent intestinal perfusion and absorption using Alexa647 conjugated AONs (HIF-1 alpha and PTEN) demonstrated significantly higher signal to noise ratio compared to FAM-conjugated AONs enabling reliable high throughput intestinal tissue perfusion and absorption detection of lower and more physiologically relevant AON concentrations. The perfusion, apical absorption and basal absorption results for the Alexa647-conjugated AONs in the subpanel of formulations are summarized in FIG. 8. Increases in intestinal absorption and perfusion (ranging from 1.3 to 3-fold compared to the non-formulated control) using Alexa647-labelled AONs were generally concordant with previously reported screening results based on FAM labelled AONs.

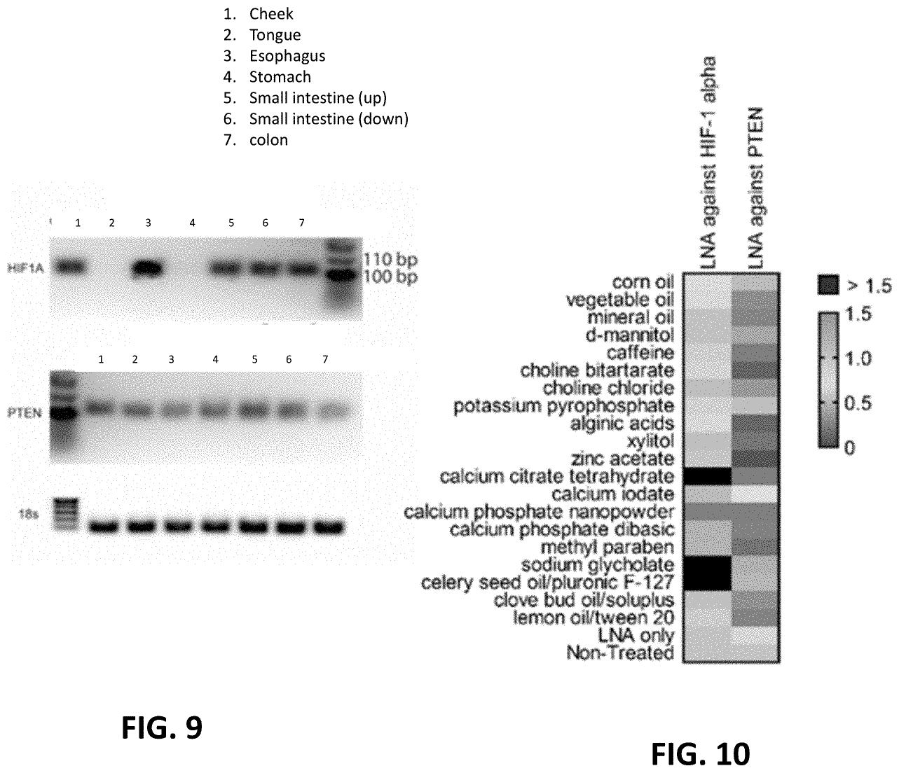

The efficacy of these AON formulations to knock-down the target gene was then examined. To measure target gene expression within the GI mucosa, methods were first developed for reproducible nucleic acid isolation from explanted GI tissue, then basal expression of the targets throughout the GI tract was quantified (FIG. 9) and quantitative rt-PCR was performed from tissue treated with the optimal formulations containing 3 μM AON. Significant knockdown was observed for the AON formulations (FIG. 10). Absolute values of expression level demonstrated that formulation-dependent changes in the target gene were not caused by effects on general expression as supported by quantitative rt-PCR of housekeeping genes.

To further characterize the delivery of the novel formulations, histological fluorescent in situ hybridization (ISH) staining was conducted of intestinal tissue cross-sections that were incubated with a subpanel of formulations and non-labelled AON against HIF-1 alpha. The results demonstrate that AON formulations enable intestinal absorption of fully intact AON whereas unformulated AON showed no signal. No interference by the formulation itself was confirmed. Interestingly, formulation dependent AON accumulation targeted to specific intestinal tissue layers was observed. In particular, AON formulations with choline bitartrate, alginic acid ammonium salt, various calcium salts, calcium phosphate nanopowder or zinc acetate showed AON accumulation limited to the epithelium while emulsion-based formulations with specific oil and emulsifier combinations appeared to enable intact AON accumulation across various intestinal layers.

Overall, the formulation dependent increase in knock-down efficiency compared to the unformulated control for non-labelled AONs against the HIF-1 alpha target is in line with the ISH histological analysis suggesting direct correlation between absorption of intact AON and knock-down efficacy. However, certain formulations appear to increase target gene expression possibly caused by effects of the formulation itself on the target gene demonstrating the importance of efficacy validation of newly identified AON formulations.

### Example 6: In Vivo Evaluation of Formulations

Based on the AON formulation validation analysis described in Example 5, formulations for AON against HIF-1 alpha were selected and the topical gastrointestinal therapeutic efficacy was tested following local GI delivery in Yorkshire pigs. In vivo evaluation of the formulations was performed through surgical access of the small intestine enabling analysis of locally administered AON formulations. Biopsy samples from the area treated were analyzed histologically by ISH staining to investigate intestinal uptake of intact AON as well as by rt-PCR to confirm activity.

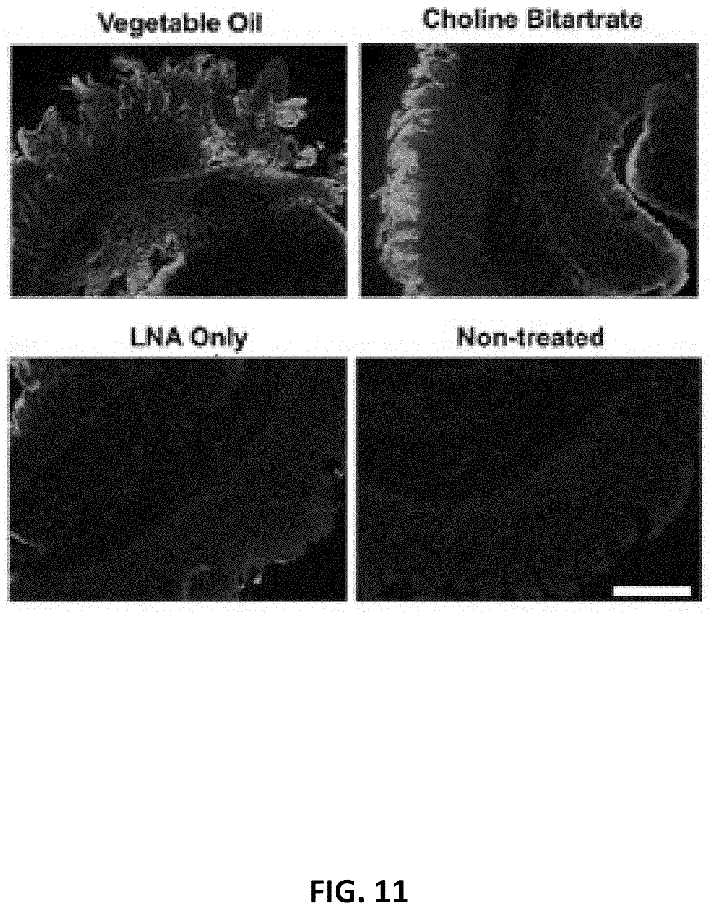

Representative ISH analysis results for intestinal uptake are shown in FIG. 11. Analysis of ISH stained histology samples showed order of magnitude increases in uptake of intact AON into various intestinal segments depending on the formulation used while non-formulated AON showed little to no absorption.

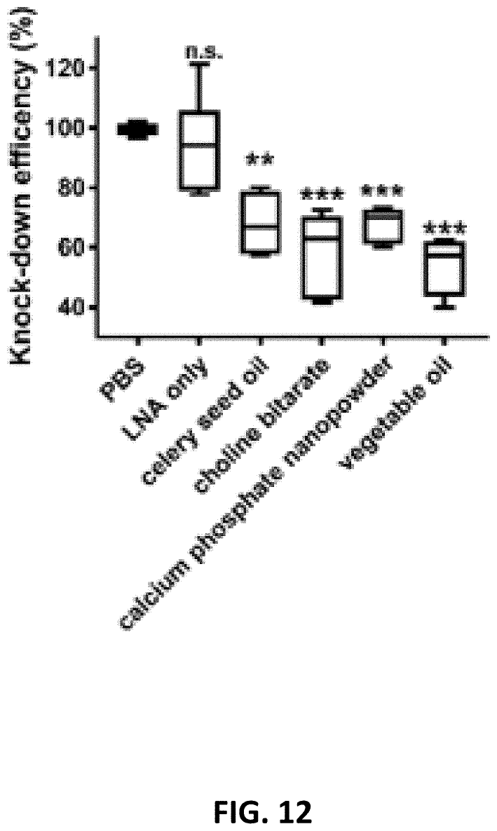

Representative expression analysis results are shown in FIG. 12. Expression analysis of the target gene demonstrated significant knock-down of the target gene across the entire tissue depth (68% for celery seed oil, 59% for choline bitatrate, 68% for calcium phosphate nanopowder and 54% for vegetable oil formulation) while non-formulated AON showed no significant effect compared to non-treated control.

Importantly, exposure of AON formulations to tissue was limited to 1 hour to approximate the short residence time of any potential oral formulation within the GI tract (Mudie et al. (2010) Mol. Pharm. 7:1388-1405). The formulations did not cause any visible histological damage to the tissue. Immunohistological analysis of in vivo biopsy samples revealed intact cell-cell adhesions after exposure to all but one AON formulation. This is particularly interesting considering that the majority of oral absorption enhancers for oligonucleotide or macromolecules in general act by disrupting the intestinal epithelial barrier function, which could raise safety concerns by the regulatory agencies (Maher et al. (2016) Adv. Drug Deliv. Rev. 106:277-319; McCartney et al. (2016) Tissue Barriers 4(2)).

Intestinal absorption enhancement of AON-formulations with the epithelial barrier function left intact, support transcellular uptake, which would explain why these formulations specifically increase AON absorption within the intestinal tissue. Interestingly, the tested AON absorption enhancers, choline bitartrate and calcium phosphate amorphous nanopowder were found to form nanoparticle aggregates with AON or emulsion-based nano- and micro particles in the case of vegetable oil emulsions. This indicates that these formulations form AON nanoparticle assemblies that enabled highly effective intestinal tissue uptake through active uptake without tissue disruption and could form the basis of a new class of highly effective oral oligonucleotide therapeutics for the effective treatment of a wide range of GI related diseases.

SEQUENCE LISTING SUMMARY

| SEQ ID | |
|---|---|
| NO: | SEQUENCE |
| 1 | 5′-GCaagcatcctGT |
| | (LNA against HIF-1 alpha) |
| 2 | 5′-[FAM]S1-GCaagcatcctGT |
| | (FAM-LNA against HIF-1 alpha) |
| 3 | 5′-TCActtagccattGGT |
| | (LNA against PTEN Version #1) |
| 4 | 5′-ACttagccatTG |
| | (LNA against PTEN Version #2) |
| 5 | 5′-[FAM]ACttagccatTG |
| | (FAM-LNA against PTEN Version #2) |
| 6 | 5′-[FAM]TCActtagccattGGT |
| | (FAM-LNA against PTEN Version #1) |
| 7 | 5′-[Alexa647]TCActtagccattGGT |
| | (Alexa647-LNA against PTEN Version #1) |
| 8 | 5′-[Alexa647]S1-GCaagcatcctGT |
| | (Alex647-LNA against HIF-1 alpha) |
| 9 | TCCAATGTTCAGTGGCGGAACTTGCAATCCTCAGTTTGTGGTCTGCCA |
| | GCTAAAGGTGAAGATATATTCCTCCAATTCAGGACCCACACGACGGG |
| | AAGACAAGTTCATGTACTTTGAGTTCCCTCAGCCATTGCCTGTGTGTG |
| | GTGACATCAAAGTAGAGTTCTTCCACAAACAGAACAAGATGCTAAAA |
| | AAGGACAAAAT |
| | (PTEN primer) |
| 10 | TATGAGCTTGCTCATCAGTTGCCACTTCCCCATAATGTGAGCTCACAT |
| | CTTGATAAGGCTTCTGTTATGAGGCTTACCATCAGCTATTTGCGTGTG |
| | AGGAAACTTCTAGATGCTGGTGATTTGGATATTGAAGATGAAATGAA |
| | GGCACAGATGAATTGTTTTTATTTGAAAGCCTTGGATGGTTTTGTTAT |
| | GGTACTCACAGATGATGGTGACATGATTTATA |
| | (HIF-1 alpha primer) |
| 11 | GTCATCCTGTTGGAGAACCTTCGCTTTCATGTGGAGGAAGAAGGGAA |
| | GGGAAAAGATGCTTCTGGGAGCAAGGTTAAAGCTGATCCAGCCAAAA |
| | TAGAAGCCTTCCGAGCTTCACTTTCCAAGCTAGGGGATG |
| | (PGK-1 primer) |

1. A composition for gastrointestinal delivery, the composition comprising: (i) at least one oligonucleotide and (ii) at least one oil, formulated as an oil emulsion, wherein gastrointestinal delivery of the composition is greater than gastrointestinal delivery of the oligonucleotide alone.

2. The composition of claim 1, which further comprises at least one emulsifier.

3. The composition of claim 1, wherein the oligonucleotide is an antisense oligonucleotide.

4. The composition of claim 3, wherein the antisense oligonucleotide is a locked nucleic acid (LNA) oligonucleotide.

5. The composition of claim 4, wherein the LNA oligonucleotide targets HIF-1 alpha or PTEN.

6. The composition of claim 1, wherein the oil is selected from the group consisting of anise oil, cade oil, canola oil, Cassia oil, castor oil, celery oil, cinnamon oil, citronella oil, clove bud oil, coconut oil, corn oil, cottonseed oil, croton oil, cypress oil, Eucalyptus oil, fennel oil, flax seed oil, geranium oil, jojoba oil, lavender oil, lemon oil, mandarin oil, mineral oil, olive oil, peanut oil, rosemary oil, sandalwood oil, soya bean oil, thyme oil, tung oil, vegetable oil, wheatgerm oil and wintergreen oil.

7. (canceled)

8. The composition of claim 2, wherein the emulsifier is selected from the group consisting of Soluplus®, Pluronic® F-127 and Tween® 20.

9. The composition of claim 1, wherein gastrointestinal absorption of the composition is greater than gastrointestinal absorption of the oligonucleotide alone.

10. The composition of claim 1, wherein gastrointestinal perfusion of the composition is greater than gastrointestinal perfusion of the oligonucleotide alone.

11. A composition for gastrointestinal delivery, the composition comprising: (i) at least one oligonucleotide; and (ii) at least one gastrointestinal delivery enhancer (GDE) selected from the group consisting of calcium salts, potassium salts, sodium salts, ammonium salts, dicarboxylic acids, cholines, chlorides, amino sugars, fatty acids, parabens, buffering agents, clays and oils, wherein gastrointestinal delivery of the composition is greater than gastrointestinal delivery of the oligonucleotide alone.

12. The composition of claim 11, wherein the GDE is:
(i) a calcium salt selected from the group consisting of calcium carbonate, calcium phosphate monobasic, calcium amorphous nanoparticles, calcium D-gluconate and alginic acid calcium;
(ii) a potassium salt selected from the group consisting of potassium phosphate dibasic and potassium disulfide;
(iii) a sodium salt selected from the group consisting of sodium metabisulfite, sodium azide, sodium perchlorate monohydrate and 3-(trimethylsilyl)-1-propanesulfonic acid sodium;
(iv) an ammonium salt, wherein the ammonium salt is ammonium iron citrate;
(v) a dicarboxylic acid, wherein the dicarboxylic acid is adipic acid;
(vi) a choline, wherein the choline is choline bitartrate;
(vii) a chloride, wherein the chloride is Tin (II) chloride;
(viii) an amino sugar, wherein the amino sugar is meglumine;
(ix) a fatty acid, wherein the fatty acid is octanoic acid or 4-ethyloctanoic acid;
(x) a paraben, wherein the paraben is methylparaben or ethyl paraben;
(xi) a buffering agent, wherein the buffering agent is HEPES or Tris base;
(xii) a clay, wherein the clay is kaolin; or
(xiii) an oil, wherein the oil is corn oil or vegetable oil.

13.-24. (canceled)

25. The composition of claim 11, wherein the oligonucleotide is an antisense oligonucleotide.

26. The composition of claim 25, wherein the antisense oligonucleotide is a locked nucleic acid (LNA) oligonucleotide.

27. The composition of claim 26, wherein the LNA oligonucleotide targets HIF-1 alpha or PTEN.

28. The composition of claim 11, wherein gastrointestinal absorption of the composition is greater than gastrointestinal absorption of the oligonucleotide alone.

29. The composition of claim 11, wherein gastrointestinal perfusion of the composition is greater than gastrointestinal perfusion of the oligonucleotide alone.

30. A method of enhancing delivery of an oligonucleotide to gastrointestinal tissue, the method comprising administering the composition of claim 1 to the gastrointestinal tissue.

31.-57. (canceled)

58. A method of enhancing delivery of a locked nucleic acid oligonucleotide that targets HIF-1 alpha to gastrointestinal tissue, the method comprising administering the composition of claim 5 to the gastrointestinal tissue.

59. A method of enhancing delivery of a locked nucleic acid oligonucleotide that targets PTEN to gastrointestinal tissue, the method comprising administering the composition of claim 5 to the gastrointestinal tissue.


SEQUENCE LISTING (11 sequences)

SEQ ID NO: 1 - DNA, 13 bp, Artificial Sequence
  Synthetic LNA against HIF-1 alpha
gcaagcatcctgt

SEQ ID NO: 2 - DNA, 13 bp, Artificial Sequence
  Synthetic FAM-LNA against HIF-1 alpha
  modified_base (1)..(1): [FAM]S1
gcaagcatcctgt

SEQ ID NO: 3 - DNA, 16 bp, Artificial Sequence
  Synthetic LNA against PTEN Version #1
tcacttagccattggt

SEQ ID NO: 4 - DNA, 12 bp, Artificial Sequence
  Synthetic LNA against PTEN Version #2
acttagccattg

SEQ ID NO: 5 - DNA, 12 bp, Artificial Sequence
  Synthetic FAM-LNA against PTEN Version #2
  modified_base (1)..(1): [FAM]
acttagccattg

SEQ ID NO: 6 - DNA, 16 bp, Artificial Sequence
  Synthetic FAM-LNA against PTEN Version #1
  modified_base (1)..(1): [FAM]
tcacttagccattggt

SEQ ID NO: 7 - DNA, 16 bp, Artificial Sequence
  Synthetic Alexa647-LNA against PTEN Version #1
  modified_base (1)..(1): [Alexa647]
tcacttagccattggt

SEQ ID NO: 8 - DNA, 13 bp, Artificial Sequence
  Synthetic Alex647-LNA against HIF-1 alpha
  modified_base (1)..(1): [Alexa647]S1
gcaagcatcctgt

SEQ ID NO: 9 - DNA, 201 bp, Artificial Sequence
  Synthetic PTEN primer
tccaatgttcagtggcggaacttgcaatcctcagtttgtggtctgccagctaaaggtgaagatatattcctccaattcaggacccacacgacgggaagacaagttcatgtactttgagttccctcagccattgcctgtgtgtggtgacatcaaagtagagttcttccacaaacagaacaagatgctaaaaaaggacaaaat

SEQ ID NO: 10 - DNA, 223 bp, Artificial Sequence
  Synthetic HIF-1 alpha primer
tatgagcttgctcatcagttgccacttccccataatgtgagctcacatcttgataaggcttctgttatgaggcttaccatcagctatttgcgtgtgaggaaacttctagatgctggtgatttggatattgaagatgaaatgaaggcacagatgaattgtttttatttgaaagccttggatggttttgttatggtactcacagatgatggtgacatgatttata

SEQ ID NO: 11 - DNA, 133 bp, Artificial Sequence
  Synthetic PGK-1 primer
gtcatcctgttggagaaccttcgctttcatgtggaggaagaagggaagggaaaagatgcttctgggagcaaggttaaagctgatccagccaaaatagaagccttccgagcttcactttccaagctaggggatg